(19) United States
(12) Patent Application Publication
(10) Pub. No.: US 2019/0333613 A1
(43) Pub. Date: Oct. 31, 2019

(54) HEALTHCARE PROVIDER-PATIENT MATCHING METHOD, SYSTEM, AND APPARATUS

(71) Applicant: RedApple Digital Health, Inc., Tustin, CA (US)

(72) Inventors: Azra Zaidi, Irvine, CA (US); Khoa Minh Huynh, Laguna Hills, CA (US)

(21) Appl. No.: 16/384,540

(22) Filed: Apr. 15, 2019

Related U.S. Application Data
(60) Provisional application No. 62/662,602, filed on Apr. 25, 2018.

(51) Int. Cl. G16H 10/60; G16H 40/63

(52) U.S. Cl. CPC G16H 10/60; G16H 40/63

(57) ABSTRACT

A method, system, and apparatus to match patients with healthcare providers are described. Patients and healthcare providers are classified into separate profile categories with similar but different information. Patients are presented an index of healthcare providers that best fit their search criteria and healthcare providers are presented an index of patient that best fit their search criteria. The patients and healthcare providers sequentially make an indication of whether they wish to match with each individual presented. When patients and healthcare providers indicate they wish to match with an individual, the individual receives a request. If the individual approves the request, a match is formed. When a patient and healthcare provider match, they are then able to communicate with one another.

### PRIORITY CLAIM

The present application claims priority to and the benefit of U.S. Provisional Patent Application No. 62/662,602 filed on Apr. 25, 2018, the entirety of which is incorporated herein by reference.

### BACKGROUND

Patient and healthcare provider satisfaction is oftentimes overlooked in the healthcare system. Generally, a healthcare system is set up for treating a patient while providing an acceptable level of comfort. Very rarely do healthcare providers determine whether a patient is satisfied or take steps to ensure they are pleased with the healthcare service they are receiving. Indeed, many healthcare providers may take offense when they are labeled as service providers.

The lack of attention paid to patient satisfaction results in medical service that, while being effective, leaves a patient feeling neglected or just part of a cog progressing through the healthcare machine. A large part of a patient's satisfaction comes from how they are paired with other healthcare providers. Oftentimes, a patient's healthcare insurance provider will dictate which healthcare provider a patient is to visit for medical service. In other instances, a patient is referred to certain healthcare providers from other healthcare providers or medical personnel. Vary rarely does a patient have the opportunity to select a healthcare provider based on their personal preferences. Indeed, a patient's selection is most often limited to healthcare provider websites that simply list bibliographic information regarding a healthcare provider. It is hard for a patient to determine if they would like a certain provider based on such impersonal information. As one can imagine, patient satisfaction with the healthcare process could improve if they are provided an opportunity to select a healthcare provider they prefer.

Similar to patients, healthcare provider satisfaction is often not considered by healthcare providers. Oftentimes, healthcare providers are assigned patients for treatment based on a scheduler. The healthcare providers virtually have no say in which patients they treat, other than for insurance reasons. This can leave healthcare providers feeling depressed, disconnected, and despondent if they have to treat patients they would prefer not to treat.

The healthcare system in the United States currently faces multiple challenges such as ever-increasing costs and uncertainties about insurance and changes in insurance programs. It is estimated that over 25 million people do not have insurance coverage. A substantial portion of the population struggles to get decent healthcare and seeks healthcare providers on their own. Searching for healthcare providers using online search engines, however, merely provides a myriad of different healthcare providers without many distinctions that individuals have to go through, which is very time consuming. Therefore, there is a need for a way for individuals to optimize their search for healthcare providers by quickly finding healthcare providers that are best suited for meeting their needs.

### SUMMARY

The presently described method, system, and apparatus are configured to match patients with healthcare providers to improve patient and healthcare provider satisfaction. Patients and healthcare providers are classified into separate categories that have similar but different attribute fields for building profiles. The information in the respective profiles is used to match patients and healthcare providers together.

Patients in the matching process are presented an index of healthcare providers that best fit the patient's search criteria. Healthcare providers in the matching process are likewise presented an index of patients that best fit the healthcare provider's search criteria. Patients and healthcare providers may then sequentially provide an indication of whether they wish to match with each of the healthcare providers or patients presented to them, respectively, or if they do not. When patients and healthcare providers indicate they wish to match with the other party, if the other party has already indicated they too wish to match, then a match is formed. If the other party has not already indicated they desire to match, then a pending request is sent to the other party. Patients and healthcare providers may view all of their pending requests and determine whether they would like to approve the pending request or reject the pending request. If they approve the pending request, a match is formed. However, if patients and healthcare providers do not approve, or do not respond to a pending request, the example system disclosed herein does not form a match.

Patients may also skip the sequential presentation of healthcare providers and locate a particular healthcare provider directly using a unique referral code that identifies the particular healthcare provider. Each healthcare provider is given a unique referral code that identifies only that healthcare provider. A patient may input a healthcare provider's code and be directed straight to that healthcare provider's profile. Healthcare providers may also share their own code with patients or other healthcare providers so others can locate the specific healthcare provider whose code was shared. Healthcare providers may have the ability to store other healthcare provider referral codes to easily share those codes with patients.

When a patient and healthcare provider match, they are then able to communicate with one another via the presently disclosed system. A patient may also directly schedule an appointment with a matched healthcare provider on the presently disclosed system. Patients and healthcare providers may also at any time decide they no longer wish to be matched with another party. Once unmatched, a patient and healthcare provider are no longer able to communicate with one another or schedule appointments on the presently disclosed system. The patient and healthcare provider, however, may match again in the future.

In light of the disclosure herein and without limiting the disclosure in any way, in a first aspect of the present disclosure, which may be combined with any other aspect listed herein unless specified otherwise, a patient-healthcare provider matching apparatus includes a processor, a patient database, a healthcare provider database, a linkage database, and a memory storing instructions. The patient database includes patient profile records for a plurality of patients, each patient profile record including patient information provided by a respective patient. The healthcare provider database includes healthcare provider profile records for a plurality of healthcare providers, each healthcare provider profile record including healthcare provider information provided by a respective healthcare provider. The linkage database includes records indicative of patient acceptances of healthcare providers, healthcare provider acceptances of patients, and linkages between mutually accepted patients and healthcare providers. The instructions stored by the memory, when executed by the processor, cause the processor to provide for matching between a patient and a healthcare provider by, for the patient, comparing the patient profile record to the healthcare provider profile records in the healthcare provider database to determine a healthcare provider index of potential matching healthcare providers, creating or accessing a healthcare provider profile card for each of the potential matching healthcare providers and determining an order for the healthcare provider profile cards based on a degree of matching with the patient profile record, with healthcare provider profile cards having a higher degree of matching being placed first in the order, causing a first ordered healthcare provider profile card to be displayed at a patient terminal of the patient, and receiving, from the patient terminal, a patient input in relation to the first ordered healthcare provider profile card. Responsive to the patient input being indicative of a rejection of the healthcare provider profile card, the processor causes a next ordered healthcare provider profile card to be displayed at the patient terminal. Responsive to the patient input being indicative of an acceptance of the healthcare provider profile card, the processor determines if the healthcare provider has already accepted the patient by accessing the linkage database. Responsive to the healthcare provider having not accepted the patient, the processor transmits a message to a healthcare provider terminal of the healthcare provider indicative of a pending request from the patient. And, responsive to the healthcare provider having already accepted the patient, the processor causes a linkage record to be created indicative of a pairing between the patient and the healthcare provider and enabling the patient and the healthcare provider to communicate with each other.

In accordance with a second aspect of the present disclosure, which may be used in combination with any other aspect listed herein unless stated otherwise, the memory stores additional instructions which, when executed by the processor, cause the processor to provide for matching between the healthcare provider and the patient by, for the healthcare provider, comparing the healthcare provider profile record to the patient profile records in the patient database to determine a patient index of potential matching patients, creating or accessing a patient profile card for each of the potential matching patients, determining an order for the patient profile cards based on a degree of matching with the healthcare provider record, with patient profile cards having a higher degree of matching being placed first in the order, causing a first ordered patient profile card to be displayed at a healthcare provider terminal of the healthcare provider, and receiving, from the healthcare provider terminal, a healthcare provider input in relation to the first ordered patient profile card. Responsive to the healthcare provider input being indicative of a rejection of the patient profile card, the processor causes a next ordered patient profile card to be displayed at the healthcare provider terminal. Responsive to the healthcare provider input being indicative of an acceptance of the patient profile card, the processor determines if the patient has already accepted the healthcare provider by accessing the linkage database. Responsive to the patient having not accepted the healthcare provider, the processor transmits a message to a patient terminal of the patient indicative of a pending request from the healthcare provider. And, responsive to the patient having already accepted the healthcare provider, the processor causes a linkage record to be created indicative of a pairing between the healthcare provider and the patient and enabling the healthcare provider and the patient to communicate with each other.

In accordance with a third aspect of the present disclosure, which may be used in combination with any other aspect listed herein unless stated otherwise, the memory stores additional instructions which, when executed by the processor, cause the processor to before or during the comparison of the patient profile record to the healthcare provider profile records in the healthcare provider database, access the linkage database to determine healthcare providers that have already rejected the patient and remove the determined healthcare providers that have already rejected the patient from being potential matching healthcare providers.

In accordance with a fourth aspect of the present disclosure, which may be used in combination with any other aspect listed herein unless stated otherwise, the memory stores additional instructions which, when executed by the processor, cause the processor to receive from the patient device a referral code corresponding to a referred healthcare provider having a healthcare provider profile record in the healthcare provider database and before or during the comparison of the patient profile record to the healthcare provider profile records in the healthcare provider database, add the healthcare provider profile record of the referred healthcare provider to the healthcare provider index regardless of a comparison of the patient profile record to the healthcare provider profile record of the referred healthcare provider. The instructions additionally cause the processor to place a healthcare provider profile card of the referred healthcare provider at a front of the order.

In accordance with a fifth aspect of the present disclosure, which may be used in combination with any other aspect listed herein unless stated otherwise, the memory stores additional instructions which, when executed by the processor, cause the processor to cause a referred designator to be displayed on the healthcare provider profile card of the referred healthcare provider.

In accordance with a sixth aspect of the present disclosure, which may be used in combination with any other aspect listed herein unless stated otherwise, the memory stores additional instructions which, when executed by the processor, cause the processor to determine at least some patient information of the patient that does not match comparable healthcare provider information of a potential matching healthcare provider and remove the non-matching healthcare provider information from the healthcare provider profile card of the potential matching healthcare provider before making the healthcare provider profile card available for display to the patient.

In accordance with a seventh aspect of the present disclosure, which may be used in combination with any other aspect listed herein unless stated otherwise, at least some of the patient information that does not match the comparable healthcare provider information corresponds to a category or a field that is coded as being non-critical.

In accordance with an eighth aspect of the present disclosure, which may be used in combination with any other aspect listed herein unless stated otherwise, the category or field is related to at least one of a language spoken, an ethnicity, a hobby, a favorite television/movie, or a social view.

In accordance with a ninth aspect of the present disclosure, which may be used in combination with any other aspect listed herein unless stated otherwise, the memory stores additional instructions which, when executed by the processor, cause the processor to create the healthcare provider profile cards by selecting a subset of the healthcare provider information for the respective healthcare provider.

In accordance with a tenth aspect of the present disclosure, which may be used in combination with any other aspect listed herein unless stated otherwise, the healthcare provider information includes a healthcare provider name, a healthcare provider picture, a healthcare provider age, a healthcare provider gender, a healthcare provider email, a healthcare provider phone number, a healthcare provider address, a healthcare provider specialization, a healthcare provider fee range, and healthcare provider accepted insurance companies, and a healthcare provider desired distance, and the healthcare provider profile card includes at least one of a healthcare provider name, a healthcare provider picture, a healthcare provider address, a healthcare provider specialization, a healthcare provider fee range, and healthcare provider accepted insurance companies.

In accordance with an eleventh aspect of the present disclosure, which may be used in combination with any other aspect listed herein unless stated otherwise, the memory stores additional instructions which, when executed by the processor, cause the processor to determine a rating for a respective healthcare provider for each of the healthcare provider profile cards and display the determined rating on the respective healthcare provider profile cards.

In accordance with a twelfth aspect of the present disclosure, which may be used in combination with any other aspect listed herein unless stated otherwise, the memory stores additional instructions which, when executed by the processor, cause the processor to at least one of either after enabling the patient and the healthcare provider to communicate with each other, provide a communication option on profile cards of the matching patient and healthcare provider that activates at least one of a chat session, a live-video session, a telecommunication session, or an email program, or after enabling the patient and the healthcare provider to communicate with each other, provide contact information on the healthcare provider profile card of the matching healthcare provider or transmit the contact information of the healthcare provider to the patient terminal.

In accordance with a thirteenth aspect of the present disclosure, which may be used in combination with any other aspect listed herein unless stated otherwise, the memory stores additional instructions which, when executed by the processor, cause the processor to after enabling the patient and the healthcare provider to communicate with each other, cause a schedule of the matching healthcare provider to be displayed on the patient terminal, receive a patient scheduling message indicative of a date and time for reserving an appointment, add an indication of the appointment to the schedule, and transmit a healthcare provider scheduling message to be transmitted to the healthcare provider terminal that is indicative of the appointment.

In accordance with a fourteenth aspect of the present disclosure, which may be used in combination with any other aspect listed herein unless stated otherwise, the memory stores additional instructions which, when executed by the processor, cause the processor to responsive to the patient input being indicative of a rejection of the healthcare provider profile card, place the healthcare provider profile card of the rejected healthcare provider into a lower position into the order.

In accordance with a fifteenth aspect of the present disclosure, which may be used in combination with any other aspect listed herein unless stated otherwise, the patient input includes at least one of a swiping motion, a selection of a graphical icon, or a gesture made by moving the patient terminal.

In accordance with a sixteenth aspect of the present disclosure, which may be used in combination with any other aspect listed herein unless stated otherwise, a patient-healthcare provider matching method includes providing a patient database including patient profile records for a plurality of patients, each patient profile record including patient information provided by a respective patient, providing a healthcare provider database including healthcare provider profile records for a plurality of healthcare providers, each healthcare provider profile record including healthcare provider information provided by a respective healthcare provider, providing a linkage database including records indicative of patient acceptances of healthcare providers, healthcare provider acceptances of patients, and linkages between mutually accepted patients and healthcare providers, and providing for matching between a patient and a healthcare provider by, for the patient, comparing, via a processor, the patient profile record to the healthcare provider profile records in the healthcare provider database to determine a healthcare provider index of potential matching healthcare providers, creating or accessing, via the processor, a healthcare provider profile card for each of the potential matching healthcare providers, determining, via the processor, an order for the healthcare provider profile cards based on a degree of matching with the patient profile record, with healthcare provider profile cards having a higher degree of matching being placed first in the order, causing, via the processor, a first ordered healthcare provider profile card to be displayed at a patient terminal of the patient, and receiving, from the patient terminal, a patient input in relation to the first ordered healthcare provider profile card. Responsive to the patient input being indicative of a rejection of the healthcare provider profile card, the method includes causing, via the processor, a next ordered healthcare provider profile card to be displayed at the patient terminal. Responsive to the patient input being indicative of an acceptance of the healthcare provider profile card, the method includes determining, via the processor, if the healthcare provider has already accepted the patient by accessing the linkage database. Responsive to the healthcare provider having not accepted the patient, the method includes transmitting, via the processor, a message to a healthcare provider terminal of the healthcare provider indicative of a pending request from the patient. Responsive to the healthcare provider having already accepted the patient, the method includes causing, via the processor, a linkage record to be created indicative of a pairing between the patient and the healthcare provider and enabling the patient and the healthcare provider to communicate with each other.

In accordance with a seventeenth aspect of the present disclosure, which may be used in combination with any other aspect listed herein unless stated otherwise, the method further includes providing for matching between the healthcare provider and the patient by, for the healthcare provider, comparing, via the processor, the healthcare provider profile record to the patient profile records in the patient database to determine a patient index of potential matching patients, creating or accessing, via the processor, a patient profile card for each of the potential matching patients, determining, via the processor, an order for the patient profile cards based on a degree of matching with the healthcare provider profile record, with patient profile cards having a higher degree of matching being placed first in the order, causing, via the processor, a first ordered patient profile card to be displayed at the healthcare provider terminal of the healthcare provider, and receiving, from the healthcare provider terminal, a healthcare provider input in relation to the first ordered patient profile card. Responsive to the healthcare provider input being indicative of a rejection of the patient profile card, the method includes causing, via the processor, a next ordered patient profile card to be displayed at the healthcare provider terminal. Responsive to the healthcare provider input being indicative of an acceptance of the patient profile card, the method includes determining, via the processor, if the patient has already accepted the healthcare provider by accessing the linkage database. Responsive to the patient having not accepted the patient, the method includes transmitting, via the processor, a message to the patient terminal of the patient indicative of a pending request from the healthcare provider. Responsive to the patient having already accepted the healthcare provider, the method includes causing, via the processor, a linkage record to be created indicative of a pairing between the patient and the healthcare provider and enabling the patient and the healthcare provider to communicate with each other.

In accordance with an eighteenth aspect of the present disclosure, which may be used in combination with any other aspect listed herein unless stated otherwise, the method further includes receiving, in the processor from the patient device, a referral code corresponding to a referred healthcare provider having a healthcare provider profile record in the healthcare provider database, before or during the comparison of the patient profile record to the healthcare provider profile records in the healthcare provider database, adding, via the processor, the healthcare provider profile record of the referred healthcare provider to the healthcare provider index regardless of a comparison of the patient profile record to the healthcare provider profile record of the referred healthcare provider, and placing, via the processor, a healthcare provider profile card of the referred healthcare provider at a front of the order.

In accordance with a nineteenth aspect of the present disclosure, which may be used in combination with any other aspect listed herein unless stated otherwise, the method further includes causing, via the processor, a referred designator to be displayed on the healthcare provider profile card of the referred healthcare provider.

In accordance with a twentieth aspect of the present disclosure, which may be used in combination with any other aspect listed herein unless stated otherwise, the method further includes after enabling the patient and the healthcare provider to communicate with each other, causing, via the processor, a schedule of the matching healthcare provider to be displayed on the patient terminal, receiving, in the processor, a patient scheduling message indicative of a date and time for reserving an appointment, adding, via the processor, an indication of the appointment to the schedule; transmitting, via the processor a healthcare provider scheduling message to be transmitted to the healthcare provider terminal that is indicative of the appointment.

In a twenty-first aspect of the present disclosure, any of the structure and functionality disclosed in connection with FIGS. 1 to 21E may be combined with any other structure and functionality disclosed in connection with FIGS. 1 to 21E.

The advantages discussed herein may be found in one, or some, and perhaps not all of the embodiments disclosed herein. Additional features and advantages are described herein, and will be apparent from, the following Detailed Description and the figures.

### BRIEF DESCRIPTION OF THE FIGURES

FIG. 1 illustrates an example embodiment of a system of the present disclosure.

FIG. 2 illustrates an example embodiment of a management server of the present disclosure.

FIGS. 3A and 3B illustrate example embodiments of registration screens of an application of the present disclosure.

FIG. 4 illustrates an example embodiment of a patient profile interface of an application of the present disclosure.

FIG. 5 illustrates an example embodiment of a healthcare provider profile interface of an application of the present disclosure.

FIGS. 6A and 6B illustrate example embodiments of methods to generate a healthcare provider index and patient index, respectively, of the present disclosure.

FIGS. 7A, 7B, and 7C illustrate an example embodiment of a method to determine a potential healthcare provider match using conditional filters of the present disclosure.

FIGS. 8A, 8B, and 8C illustrate example embodiments of a healthcare provider profile card of an application of the present disclosure.

FIGS. 9A, 9B, 9C, and 9D illustrate example embodiments of a patient profile card of an application of the present disclosure.

FIGS. 10A and 10B illustrate example embodiments of a method for accepting or declining profile cards of the present disclosure.

FIGS. 11A, 11B, and 11C illustrate example embodiments of a method for dynamically ordering a healthcare provider index of the present disclosure.

FIG. 12 illustrates an example embodiment of a method for approving or rejecting requests of the present disclosure.

FIG. 13 illustrates an example embodiment of a method for ordering a patient index in response to a patient entering a referral code of the present disclosure.

FIG. 14 illustrates an example embodiment of a patient referral screen of an application of the present disclosure.

FIG. 15 illustrates an example embodiment of a profile card of a patient who has been referred of an application of the present disclosure.

FIG. 16 illustrates an example embodiment of a healthcare provider referral code screen of an application of the present disclosure.

FIG. 17 illustrates an example embodiment of a saved healthcare provider referral code screen of an application of the present disclosure.

FIG. 18 illustrates an example embodiment of a healthcare provider profile card of an application of the present disclosure for a healthcare provider that has actively referred other healthcare providers.

FIGS. 19A, 19B, and 19C illustrate example embodiments of healthcare provider availability screens of an application of the present disclosure.

FIGS. 20A and 20B illustrate example embodiments of patient scheduling screens of an application of the present disclosure.

FIGS. 21A, 21B, 21C, 21D, and 21E illustrate example embodiments of test scheduling screens of an application of the present disclosure.

### DETAILED DESCRIPTION

The present disclosure relates in general to a method, system, and apparatus configured to improve patient and healthcare provider satisfaction and engagement by automatically providing for the matching of patients with healthcare providers. In particular, the example method, system, and apparatus use at least one of an algorithm, machine learning routine, conditional filter(s), and/or processes to determine potential matches between patients and healthcare providers based on one or more attributes, properties, and/or preferences (e.g., information). The example method, system, and apparatus may weight or otherwise order potential matches based on one or more criteria.

Potential matches of healthcare providers are displayed by the example method, system, and apparatus for selection by a patient. In addition, potential matches of patients are displayed by the example method, system, and apparatus for selection by a healthcare provider. If a patient accepts a healthcare provider or a healthcare provider accepts a patient, the example method, system and apparatus sends a request to the accepted party who may approve (e.g., accept) or reject (e.g., decline) the request. A match is made by the example method, system, and apparatus if a patient or healthcare provider approves a request. After a match is made, the example method, system, and apparatus configures one or more communicative features on a patient's terminal and/or a healthcare provider's terminal to enable communication therebetween. The example method, system, and apparatus may also make a healthcare provider's calendar available to enable a patient to make an appointment.

It should be understood that while the presently disclosed method, system, and apparatus is described as being used only by patients and healthcare providers, other parties may use the presently disclosed method, system, and apparatus to generate matches on their behalf. For example, in the case of healthcare providers, medical staff authorized to perform tasks for the healthcare provider may use the method, system, and apparatus on behalf of the healthcare provider. In the case of patients, a family member may be authorized by the patient to use the method, system, and apparatus, on the patient's behalf. In some instances, automatons or bots may also be used to act on behalf of a patient or healthcare provider.

Reference is made herein to healthcare providers. As disclosed herein, healthcare providers may include doctors (including primary care and specialists), ophthalmologists, dermatologists, surgeons, pediatricians, nurse practitioners, chiropractors, therapists, physical therapists, dentists, psychologists, psychiatrists, nutritionists, yoga instructors, or any other provider of health or wellness services. It should be appreciated that while healthcare providers are disclosed, the example method, system, and apparatus may provide matches between consumers and other types of service providers, such as home repair individuals, mechanics, veterinarians, etc.

The example system disclosed herein enables a patient to select one or more healthcare providers or disciplines for potential engagement. This enables, for example, a patient to connect with a complete set of healthcare providers through a single system without having to conduct painstaking research into different healthcare areas or be limited to providers of a single healthcare network. For instance, a patient may use the example system to select, at the same time, a cardiologist, an endocrinologist, a diagnostic healthcare provider, an obstetrician, an immunologist, and an orthopedist.

### System Embodiment

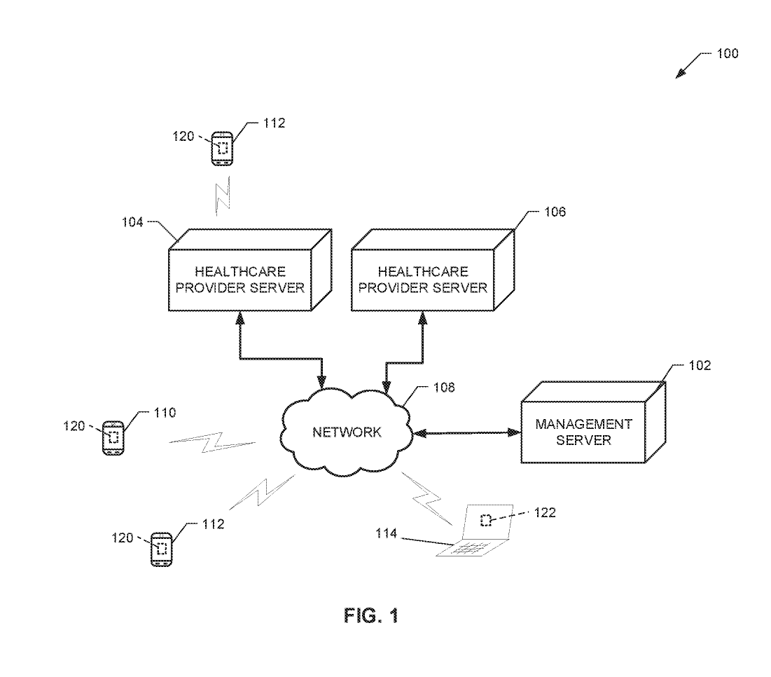

FIG. 1 shows an example embodiment of a system 100 that provides for matches between healthcare providers and patients, according to an example embodiment of the present disclosure. The system 100 may include a management server 102 communicatively coupled to one or more patient terminal 110 and one or more healthcare provider server 104 and 106 via a network 108 (e.g., the Internet). The example management server 102 is configured to provide matches between patients of the patient terminals 110 and healthcare providers associated with the healthcare provider servers 104 and 106. A healthcare provider may access the management server 102 through one of the healthcare provider servers 104 and 106 via a healthcare provider terminal 112. In other examples, the healthcare provider terminal 112 may connect directly to the network 108, bypassing the servers 104 and 106.

The patient terminal 110 and/or the healthcare provider terminal 112 may include any type of device including a smartphone, a cellular phone, a tablet computer, a laptop computer 114 (FIG. 1), a workstation, smart-eyewear, smartwatch, etc. The servers 104 and 106 may include any healthcare computer system and/or network, such as an enterprise system. The healthcare provider servers 104 and 106 may maintain patient schedules for associated healthcare providers.

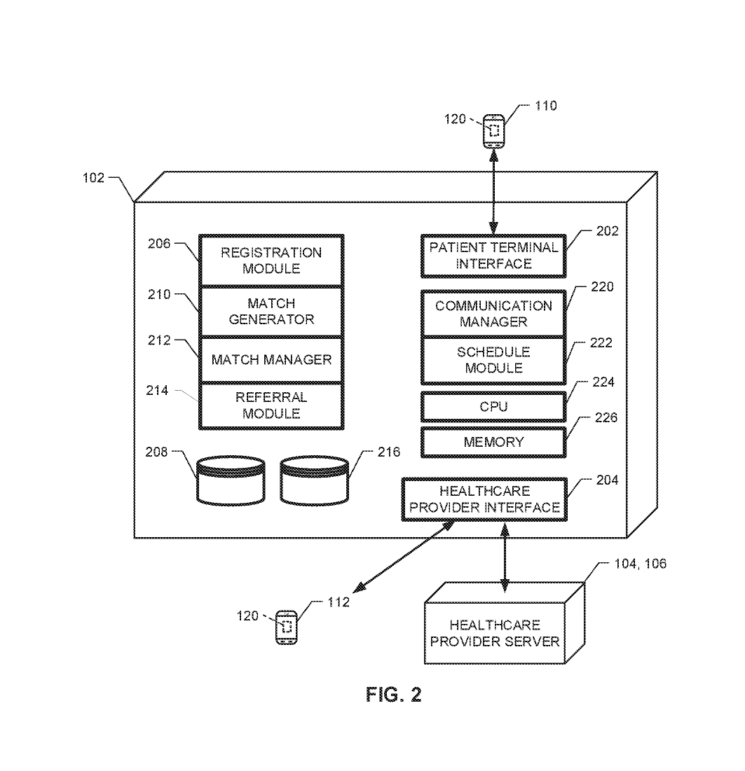

The example patient terminal 110 and/or the healthcare provider terminal 112 includes an application 120 (e.g., an App). The example application 120 is configured to acquire registration information, display indications of potential matches, record responses to the potential matches, and provide communication between a patient and a healthcare provider. The application 120 may operate in connection with the management server 102, which determines and orders potential matches between patient and healthcare providers. The management server 102 may also be configured to facilitate communication between a patient and healthcare provider after a match has been made. Further information regarding the application 120 and the management server 102 are discussed in more detail in connection with FIGS. 2 to 20.

Throughout this disclosure, various examples of the application 120 are discussed to explain the presently disclosed method, system, and apparatus. It should be appreciated, however, that in various embodiments the application 120 may be in addition to, or replaced by, a website 122 hosted or otherwise provided by the management server 102. In these embodiments, patients and healthcare providers may use respective terminals 110 and 112 to access the website 122 of the management server 102. A web browser on the terminals 110 and 112 is used to access the website for providing registration information, displaying indications of potential matches, recording responses to the potential matches, and providing communication between a patient and a healthcare provider. For example, FIG. 1 illustrates an embodiment in which a laptop computer 114 is used to access a website 122 that is managed by the management server 102.

FIG. 2 shows an example diagram of the management server 102 of FIG. 1, according to an example embodiment of the present disclosure. The management server 102 includes different components that are representative of computational processes, routines, and/or algorithms. In some embodiments, the computational processes, routines, and/or algorithms may be specified in one or more instructions stored on a computer readable medium that, when executed by a processor of the management server 102, cause the management server 102 to perform the operations discussed below. For example, all or part of the computational processes, routines, and/or algorithms may be implemented by the CPU 224 and the memory 226. It should be appreciated that in other embodiments the components of the management server 102 may be combined, rearranged, removed, or provided on a separate device or server.

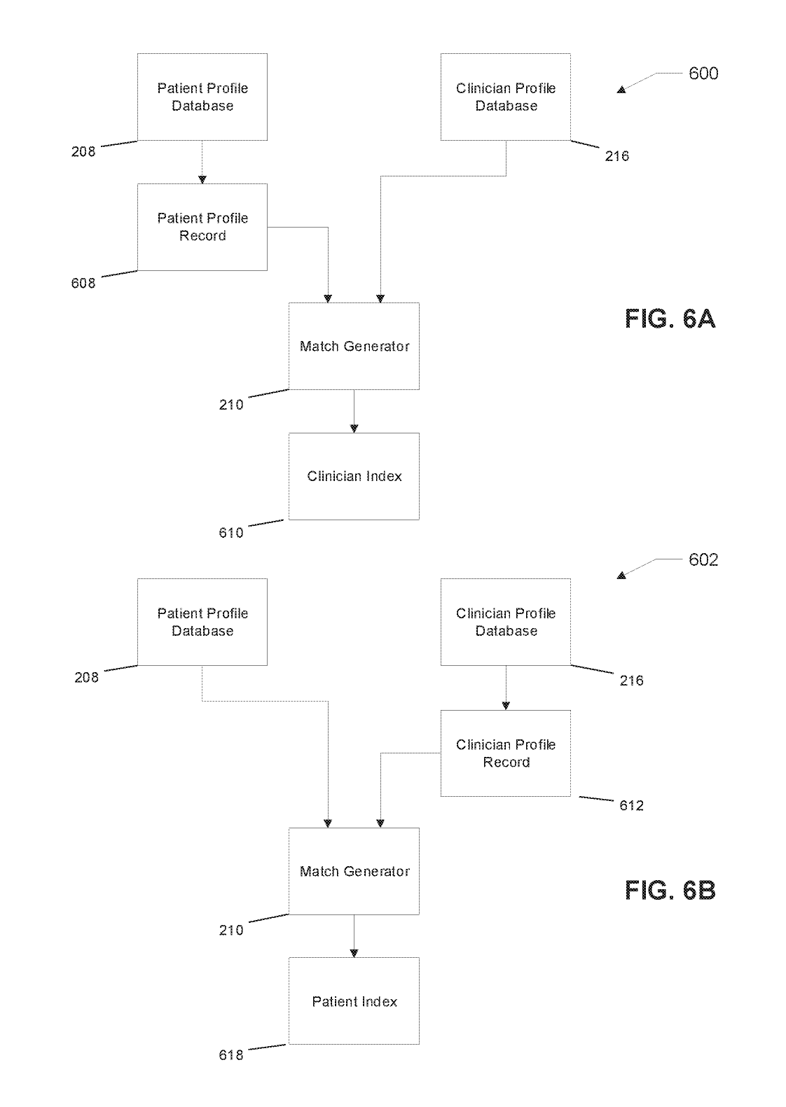

The example management server 102 includes a patient terminal interface 202 configured to be communicatively coupled to one or more patient terminal 110. The patient terminal interface 202 is configured to facilitate communications between the patient terminals 110 and the management server 102. For example, the patient terminal interface 202 may convert messages received from the patient terminal 110 into a format compatible with the management server 102. The patient terminal interface 202 may also include one or more application programming interfaces (“APIs”) that operate in connection with the application 120 for receiving data. The APIs may be configured for registration to complete a patient profile record 608 (FIG. 6A). The APIs may also be configured for displaying information indicative of potential matching healthcare providers through the application 120 and receiving responses to the potential matches.

The example management server 102 may also include a healthcare provider interface 204 that is communicatively coupled to the servers 104 and 106 and/or the healthcare provider terminal 112. Similar to the patient terminal interface 202, the healthcare provider interface 204 may be configured to format incoming and outgoing data in addition to providing one or more APIs for registration and/or matching.

The management server 102 of FIG. 2 may include a registration module 206 configured to create patient profile records 608 based on patient registration information and healthcare provider profile records 612 (FIG. 6B) based on healthcare provider registration information. The patient profile records 608 may be stored in a patient profile database 208 and the healthcare provider profile records 612 may be stored in a healthcare provider profile database 216 or on the healthcare provider servers 104 and 106. The profile records 608 and 612 are used for determining potential matches between healthcare providers and patients. To determine potential matches between patients and healthcare providers, the example management server 102 may include a match generator 210. The example match generator 210 may be configured to operate according to one or more algorithms (e.g., a machine learning algorithm), routines, conditional filter(s), etc. to determine potential matches between patients and healthcare providers. The match generator 210 may also be configured to track which profiles have been displayed to the patients and healthcare providers and records their respective responses. The match generator 210 may also be configured to track pending requests and transmit matches to a match manager 212 (introduced below) as they occur.

The example registration module 206 may also be configured for installation and/or configuration of the application 120 at the patient and healthcare provider terminals 110 and 112. In an example, a patient uses patient terminal 110 to send a message (via text, email, website etc.) to the management server 108 with a phone number and/or email address. In response, the registration module 206 transmits to the patient terminal 110 an installation file for installing the application 120 and/or a verification code. The patient provides the verification code before and/or during registration to have their information stored in a patient profile record 608. In other examples, after receiving the request from the patient, the registration module 206 transmits a message with a verification code. The patient then downloads the application 120 from a third-party site and uses the verification code to activate or otherwise gain access to the application 120.

The example management server 102 of FIG. 2 may also include a match manager 212 that records and manages matches between patients and healthcare providers as they are made. The match manager 212 may also be configured to enable or disable communication between matched patients and healthcare providers. It should be appreciated that the functions of match generator 210 and match manager 212 may be interchangeable. It should also be appreciated that in some embodiments the match generator 210 and match manager 212 are a single component that performs the functions of both as described herein. The example management server 102 may also include a referral module 214 that is configured to carry out the referral system for generating matches described in more detail below.

The example management server 102 may additionally include a communication manager 220 that enables patients and healthcare providers to communicate with each other once a match is made, for example, via the application 120 on their respective terminals 110 and 112.

Further, the example management server 102 may include a schedule module 222 that is configured to enable a matched patient to schedule an appointment with a healthcare provider or a medical diagnostic test with a test provider. Further detail regarding the components of the example management server 102 and their respective functions is provided below.

### Registration Embodiment

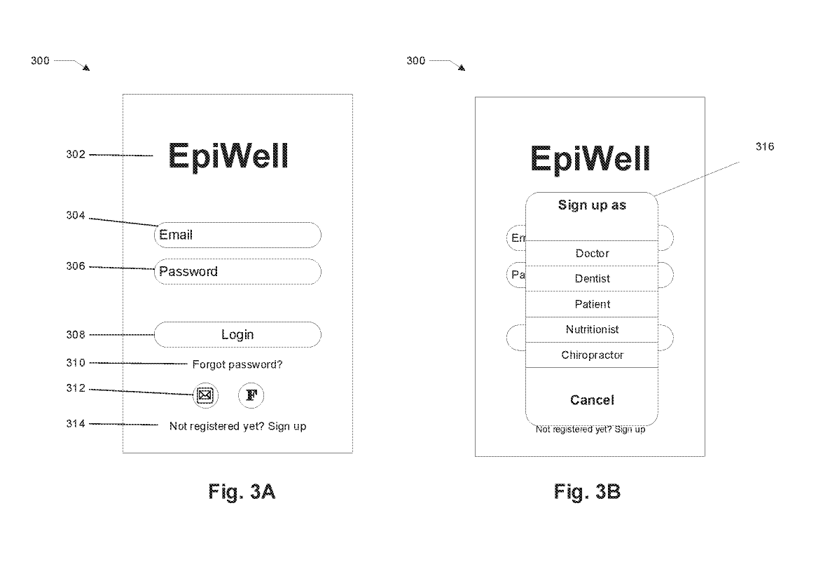

As discussed above, the management server 102 of FIGS. 1 and 2 may include a registration module 206 configured to create patient profile records 608 (FIG. 6) based on patient registration information and healthcare provider profile records 612 (FIG. 6) based on healthcare provider registration information. The example application 120 may be configured with a user interface or home screen 300 such as the one illustrated in FIG. 3A with a logo 302 located near the top of the interface. If a patient or healthcare provider has already registered a profile record 608 or 612, the patient or healthcare provider may input their login information into a username field 304 and a password field 306 and press a login button 308 to access the example application 120. If a patient or healthcare provider does not remember their login information they may press the forgot password button 310 to recover or reset their login information. In various embodiments, patients and healthcare providers may also utilize one of a plurality of share buttons 312 to share a link to the application 120 with other people, for example, over social media or email.

If a patient or healthcare provider has not already registered a profile record 608 or 612, the patient or healthcare provider may press the registration button 314. In at least one embodiment, the registration button 314 will direct a patient or healthcare provider to a user interface such as sign up screen 316 in FIG. 3B at which the patient or healthcare provider must choose a category for registration. The categories may include patient and the various types of applicable healthcare providers previously described in more detail. The registration module 206 may register a patient and healthcare provider in the same manner; however, in some embodiments, if a healthcare provider category is chosen, the registration module 206 may prompt the healthcare provider for medical licenses. The registration module 206 may then verify the medical licenses by accessing an appropriate medical licensing entity. In some embodiments, after the licenses are verified, the registration module 206 may provide a verification code for installing or registering or using the application 120. In other embodiments, a healthcare provider may be able to proceed to create a profile record 612 without a verification code. In other embodiments still, the registration module 206 may not prompt the healthcare provider for medical licenses until later in the registration process.

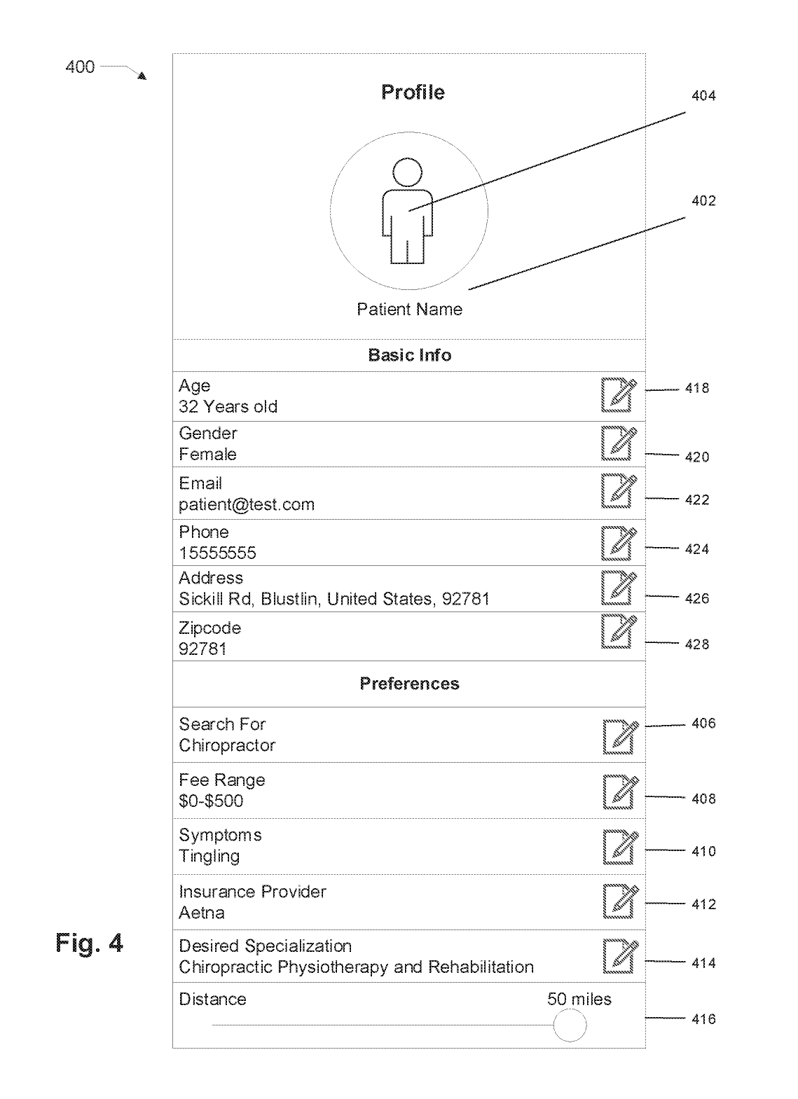

During registration, patients and healthcare providers use terminals 110 and 112 to provide information for their profile record 608 or 612 respectively. The example application 120 may be configured with a form or user interface with data fields that patients and healthcare providers populate to provide their attributes, properties, and/or preferences. FIG. 4 illustrates an example embodiment of a patient profile interface 400, according to an example embodiment of the present disclosure. The example patient profile interface 400 includes a patient name field 402, a patient picture field 404, a patient age field 418, a patient gender field 420, a patient email field 422, a patient phone number field 424, a patient address field 426, a patient zip code field 428, a desired healthcare provider type field 406, a patient fee range field 408, a patient symptoms field 410, a patient insurance field 412, a desired healthcare provider specializations field 414, and a patient desired distance field 416.

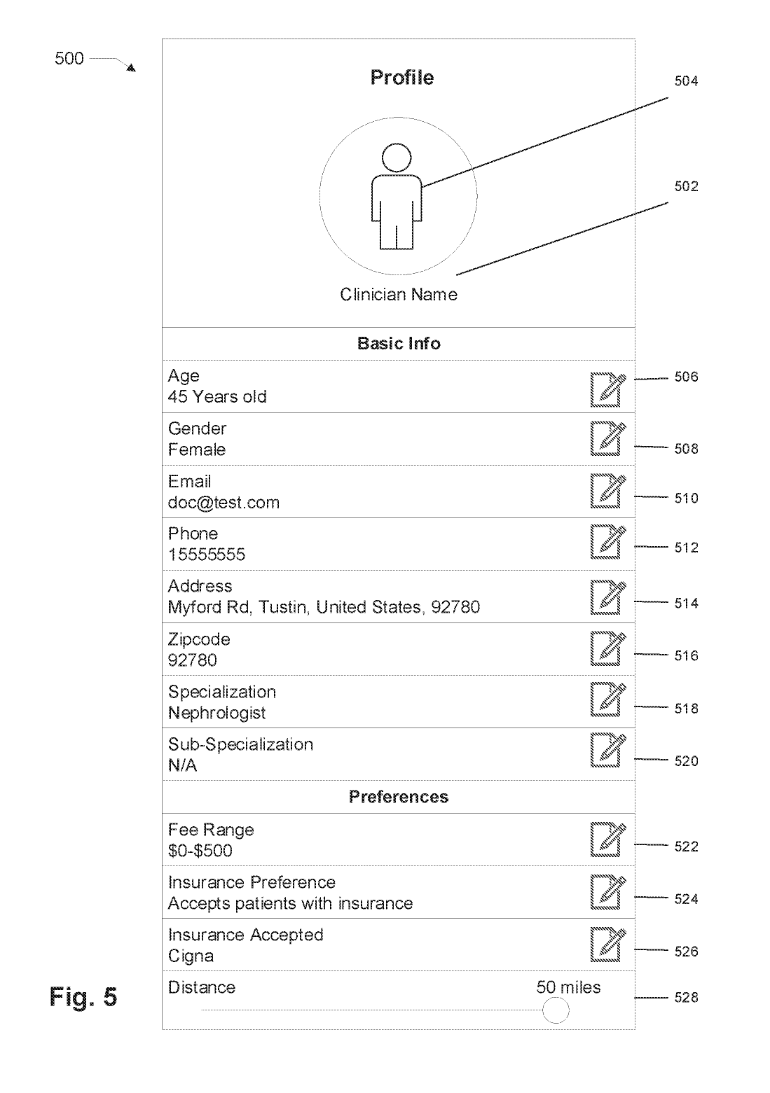

FIG. 5 illustrates an example embodiment of a healthcare provider profile interface 500, according to an example embodiment of the present disclosure. The example healthcare provider profile interface 500 includes a healthcare provider name field 502, a healthcare provider picture field 504, a healthcare provider age field 506, a healthcare provider gender field 508, a healthcare provider email field 510, a healthcare provider phone number field 512, a healthcare provider address field 514, a healthcare provider zip code field 516, a healthcare provider specialization field 518, a healthcare provider sub-specialization field 520, a healthcare provider fee range field 522, a healthcare provider insurance preference field 524, a healthcare provider accepted insurance companies field 526, and a healthcare provider desired distance field 528. The mileage indicated in the healthcare provider desired distance field 528 may be a radius of miles from the healthcare provider's location that the healthcare provider would prefer to be shown potential matching patients within.

It should be appreciated that the information included in the example patient profile interface 400 and healthcare provider profile interface 500 is not exhaustive. The information may include insurance information, pricing preferences, affordability preferences, availability preferences (e.g., times/days of the week the patient is available for an appointment), a rating preference, medical symptoms or reasons care is being sought, a type of healthcare provider being sought, a medical specialization, alternative medication preference, a geographic location, a gender, a weight, an age, languages spoken, etc. Preferences may also include patient personal preferences (as input from a patient) including personality preference for a healthcare provider and other factors that may provide for a personality match (e.g., favorite movies/television shows, hobbies, vacation locations, social views, etc.).

It should also be appreciated that at least some of the information provided by patients is not displayed to healthcare providers. Instead, the information is only used for matching. For example, the social views may never be displayed but are used to match patients and healthcare providers with similar social outlooks. Thus, the patient and healthcare provider are not provided social views of the other but are matched for having the same views. Additionally or alternatively, at least some of the preference or other information fields may be designated as being non-critical such that if the preference or information does not match corresponding preference or information for a healthcare provider (or a patient for healthcare provider preferences), the preference/information is not displayed on a version or instance of the healthcare provider's profile card that is provided to that patient. In some embodiments, a patient or healthcare provider may leave certain fields blank or empty. In these embodiments, the registration module 206 may provide an entry of “no preference” for that category when making a match. Certain data fields may be required, such as geography, before the registration module 206 enables a patient or healthcare provider to register.

In some embodiments, the registration module 206 may prompt a patient and/or healthcare provider for a picture, a video, or an advertisement. In other instances, the application 120 may provide a prompt. Selection of the prompt may open a camera application on the patient or healthcare provider terminal 110 or 112 to enable the patient or healthcare provider to record an image or video of themselves. Selection of the prompt may alternatively enable a patient or healthcare provider to select an image file to provide for their profile record 608 or 612.

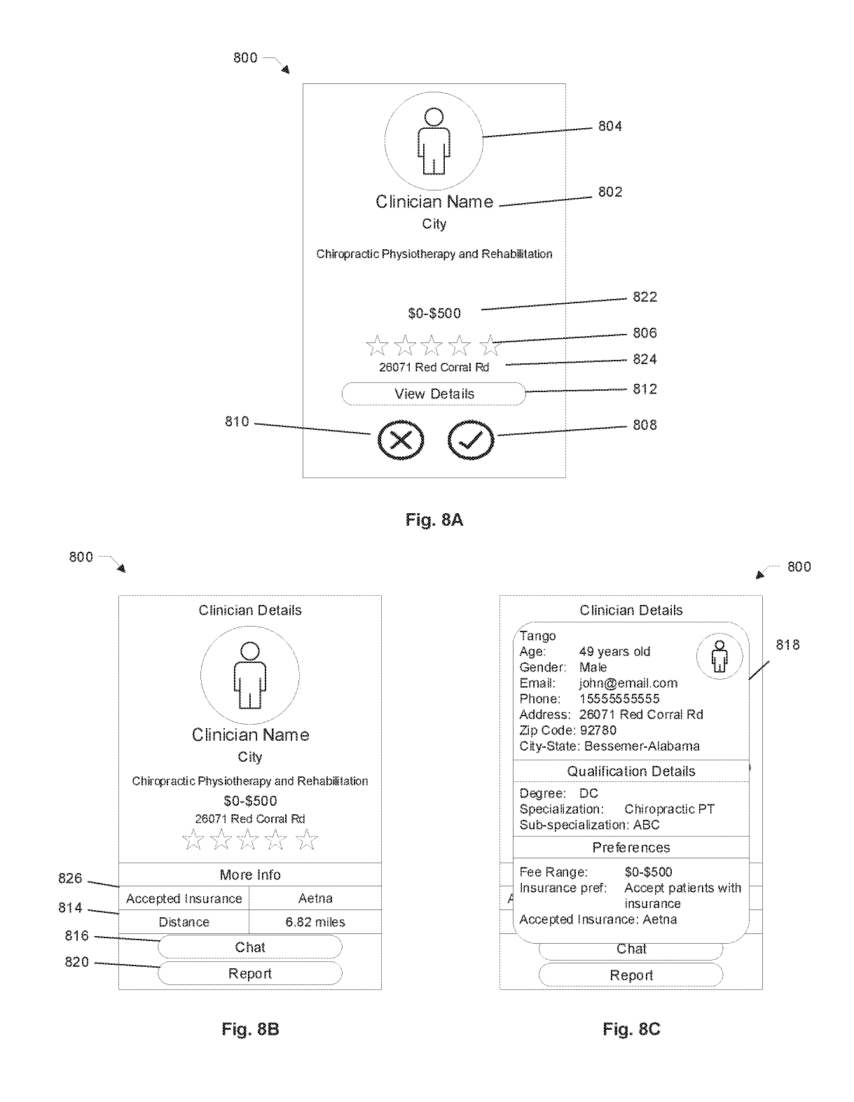

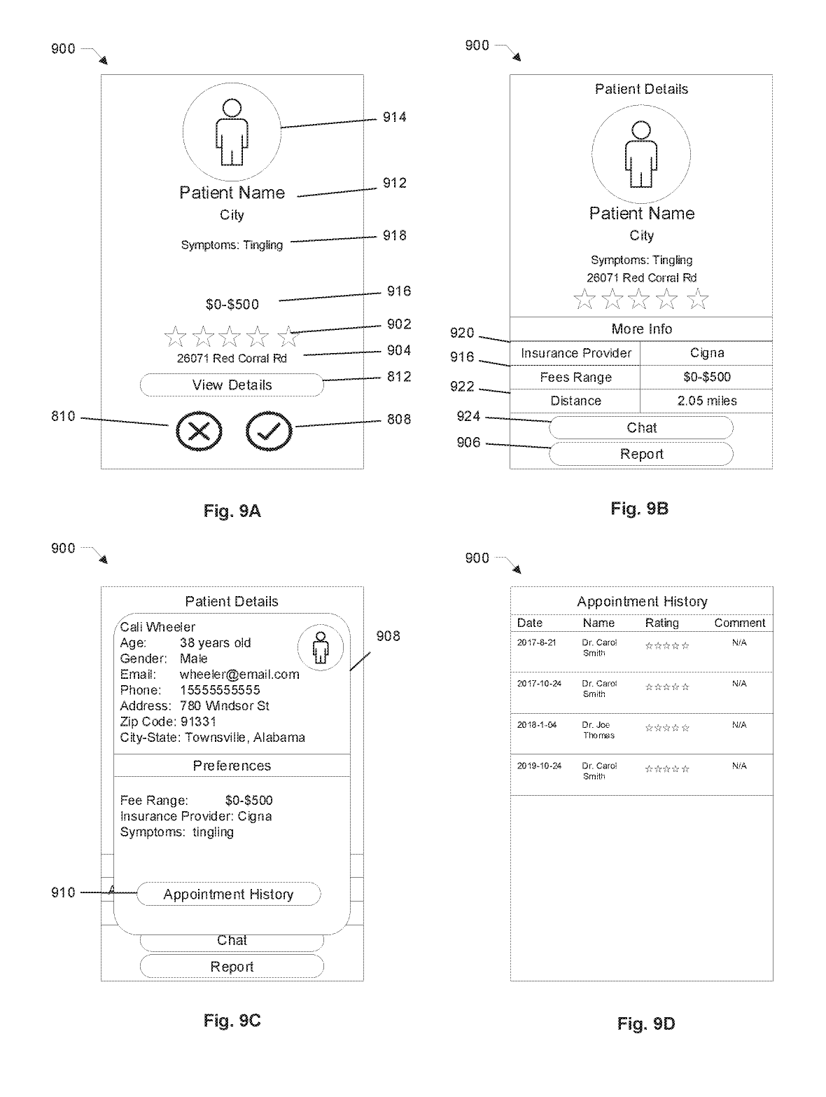

After receiving the patient and/or healthcare provider information, the registration module 206 may create a corresponding profile record 608 or 612, with patient profile records 608 stored in patient profile database 208 and healthcare provider profiles 612 stored in healthcare provider profile database 216. A match generator 210 may utilize the patient and healthcare provider profile records 608 and 612 to generate matches; however, the patient and healthcare provider profile records 608 and 612 themselves are not provided for display on application 120. Instead, the registration module 206 creates a patient profile card 900 (FIG. 9) and a healthcare provider profile card 800 (FIG. 8) for each patient and healthcare provider, which the application 120 displays when patients and healthcare providers browse potential matches. Patient and healthcare provider profile cards 900 and 800 will be discussed in more detail below.

### Patient and Healthcare Provider Indexes Embodiment

In various embodiments, the example management server 102 includes a match generator 210 to determine potential matches between patients and healthcare providers. The example match generator 210 may be configured to operate according to one or more algorithms (e.g., a machine learning algorithm), routines, conditional filter(s), etc. to determine potential matches between patients and healthcare providers. FIGS. 6A and 6B illustrate example embodiments of methods 600 and 602 to determine a healthcare provider index 610 of potential matches for patients and a patient index 618 of potential matches for healthcare providers, respectively. As discussed in more detail below, patients view healthcare provider profile cards 800 generated from the healthcare provider index 610 and indicate whether to accept (e.g., approve) or decline (e.g., reject) the healthcare provider corresponding to each healthcare provider profile card 800. Healthcare providers do the same for patient profile cards 900 generated from the patient index 618.

The methods 600 and 602 may be implemented on a computer system, such as the management server 102, the patient terminal 110, and/or the healthcare provider terminal 112. For example, the method 1300 may be implemented by the registration module 206, the match generator 210, the match manager 212, and/or the referral module 214 of the management server 102. The methods 600 and 602 may also be implemented by a set of instructions stored on a computer readable medium that, when executed by a processor, cause the computer system to perform the method. For example, all or part of the methods 600 and 602 may be implemented by the CPU 224 and the memory 226. Although the examples below are described with reference to the flowchart illustrated in FIGS. 6A and 6B, many other methods of performing the acts associated with FIGS. 6A and 6B may be used. For example, the order of some of the blocks may be changed, certain blocks may be combined with other blocks, one or more of the blocks may be repeated, and some of the blocks described may be optional.

Referring to the method 600 in FIG. 6A, to generate a healthcare provider index 610, the match generator 210 may compare a single patient profile record 608 from the patient profile database 208 with the plurality of healthcare provider profile records 612 in the healthcare provider database 216. Conversely, in the method 602 in FIG. 6B, to generate a patient index 618, the match generator 210 may compare a single healthcare provider profile record 612 from the healthcare provider profile database 216 with the plurality of patient profile records 608 in the patient profile database 208. When comparing a patient profile record 608 with a plurality of healthcare provider profile records 612, or a healthcare provider profile record 612 with a plurality of patient profile records 608, match generator 210 may compare the information in the respective records 608 and 612 according to one or more algorithms (e.g., a machine learning algorithm), routines, conditional filter(s), etc. to determine the healthcare provider index 610 of potential matches for a patient or the patient index 618 of potential matches for a healthcare provider.

For example, if a patient requests a cardiologist, only cardiologists are selected, at least initially, as potential matches. Each different conditional filter may remove additional healthcare provider profile records 612 such that after passing through the conditional filters, the remaining healthcare provider profile records 612 should at least meet the search requirements of the patient.

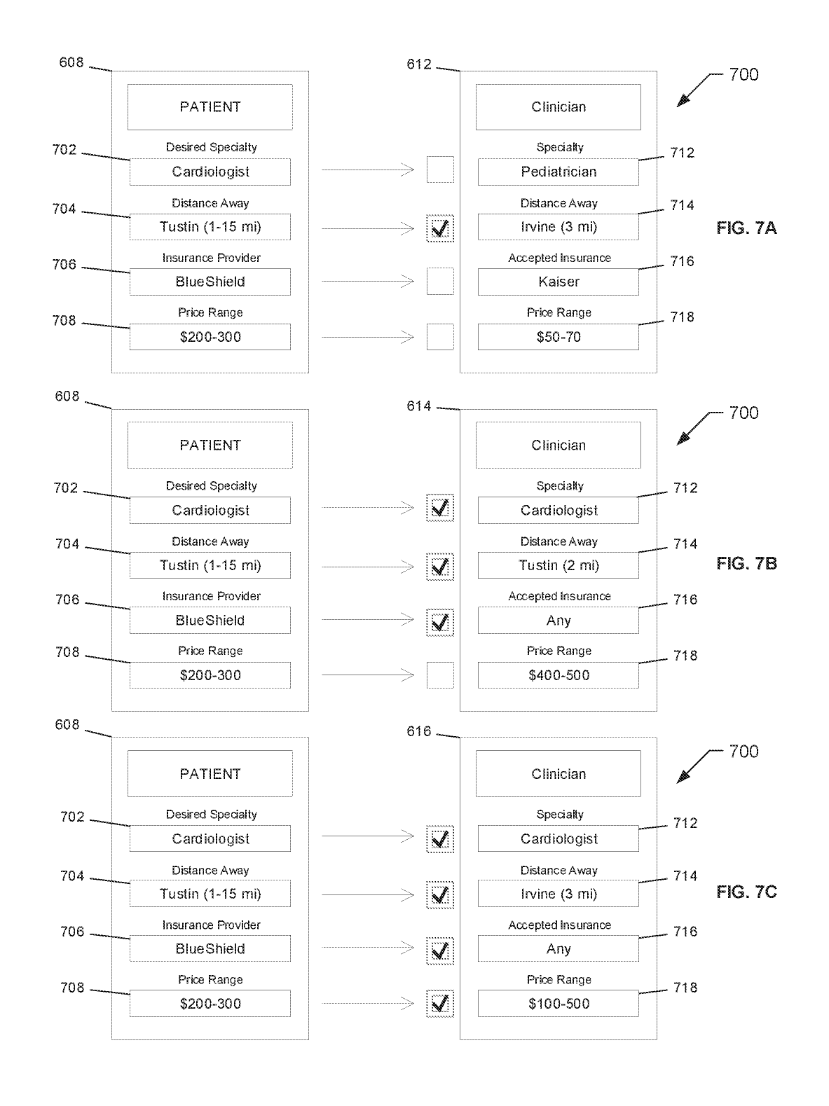

FIGS. 7A, 7B, and 7C illustrate an example embodiment of a method 700 in which the match generator 210 operates according to conditional filters to generate a healthcare provider index 610. The method 700 may be implemented on a computer system, such as the management server 102, the patient terminal 110, and/or the healthcare provider terminal 112. For example, the method 1300 may be implemented by the registration module 206, the match generator 210, the match manager 212, and/or the referral module 214 of the management server 102. The method 700 may also be implemented by a set of instructions stored on a computer readable medium that, when executed by a processor, cause the computer system to perform the method. For example, all or part of the method 700 may be implemented by the CPU 224 and the memory 226. Although the examples below are described with reference to the flowchart illustrated in FIG. 7, many other methods of performing the acts associated with FIG. 7 may be used. For example, the order of some of the blocks may be changed, certain blocks may be combined with other blocks, one or more of the blocks may be repeated, and some of the blocks described may be optional.

In the example embodiment of the method 700, match generator 210 may remove healthcare provider profile records 612 that do not provide a potential match for a patient. For example, match generator 210 first compares a patient profile record 608 with a healthcare provider profile record 612. In the example embodiment, the patient profile record 608 includes information for a desired healthcare provider specialty 702, a distance away 704, an insurance provider 706, and a price range 708. The match generator 210 may compare the information in the patient profile record 608 with the corresponding information in the healthcare provider profile record 612. It should be appreciated, however, that the patient profile record 608 and the healthcare provider profile record 612 may include any of the information previously discussed.

For the healthcare provider profile record 612, the healthcare provider's specialty 712 does not match the patient's desired healthcare provider specialty 702, indicated by the empty check box in FIG. 7A. The healthcare provider's distance away 714 matches the patient's distance away 704, indicated by the check in FIG. 7A. The healthcare provider's accepted insurance 716 does not match the patient's insurance provider 706. And the healthcare provider's price range 718 does not match the patient's price range 708. In various embodiments, because only one category of information matched between the patient and healthcare provider, the match generator 210 may not add the healthcare provider profile record 612 to the healthcare provider index 610.

Continuing in the example embodiment of the method 700, the match generator 210 may then compare the patient profile record 608 with a second healthcare provider profile record 614, as illustrated in FIG. 7B. For the second healthcare provider profile record 614, the healthcare provider's specialty 712 matches the patient's desired healthcare provider specialty 702. The healthcare provider's distance away 714 matches the patient's distance away 704. The healthcare provider's accepted insurance 716 matches the patient's insurance provider 706. However, the healthcare provider's price range 718 does not match the patient's price range 708. In various embodiments, because only three categories of information, and not all four, matched between the patient and healthcare provider, the match generator 210 may not add the second healthcare provider profile record 614 to the healthcare provider index 610. In other embodiments, three out of four matching categories of information may be sufficient for the match generator 210 to add the second healthcare provider profile record 614 to the healthcare provider index 610. It should be appreciated that the threshold of sufficient matching information for a healthcare provider profile record 612 to be added to the healthcare provider index 610 may be based on a number of matching categories, a percentage of matching categories, an algorithm, or any other method of determining a potential match with requested information. In some embodiments, at least some categories may be classified or coded as being critical while at least some other categories are classified or coded as being non-critical. The example management server 102 is configured to prevent a match from occurring between a patient and a healthcare provider if, for example, there is not a match for any or all of the designated critical categories.

Continuing further in the example embodiment of the method 700, the match generator 210 may then compare the patient profile record 608 with a third healthcare provider profile record 616, as illustrated in FIG. 7C. For the third healthcare provider profile record 616, the healthcare provider's specialty 712 matches the patient's desired healthcare provider specialty 702. The healthcare provider's distance away 714 matches the patient's distance away 704. The healthcare provider's accepted insurance 716 matches the patient's insurance provider 706. And the healthcare provider's price range 718 matches the patient's price range 708. In the example embodiment, because all four categories of information matched, the match generator may add the third healthcare provider profile record 616 to the healthcare provider index 610. It should be understood that the method 700 is repeated until the match generator 210 compares the patient profile record 608 with every healthcare provider profile record 612, 614, 616 in the healthcare provider profile database 216 to generate the healthcare provider index 610. It should also be appreciated that the method 700 is performed in the same manner to generate the patient index 618 by comparing a healthcare provider profile record 612 with every patient profile record 608 in the patient profile database 208.

In some embodiments, the match generator 210 may compare other attributes, properties, and/or preferences in patient profile record 608 with every healthcare provider profile record 612, 614, 616 to determine the order of the healthcare provider profile records 612 in the healthcare provider index 610. For example, the match generator 210 may use conditional filters as described above to determine a set of potential matches between a patient and healthcare providers and then the match generator 210 may use matches of other attributes, properties, and/or preferences to order the healthcare providers. In some example embodiments, the match generator 210 may assign different weights to attributes, properties, and/or preferences or provide a match weight based on a closeness of the information. The match generator 210 may then calculate a total score for each healthcare provider profile record 612, which is used for ordering, ranking, or filtering above a predetermined threshold. In other embodiments, the match generator 210 may employ other methods or formulas of ordering healthcare provider profile records 612. It should again be appreciated that the embodiments including ordering, ranking, or filtering also apply to the patient index 618.

### Match Generation Embodiment

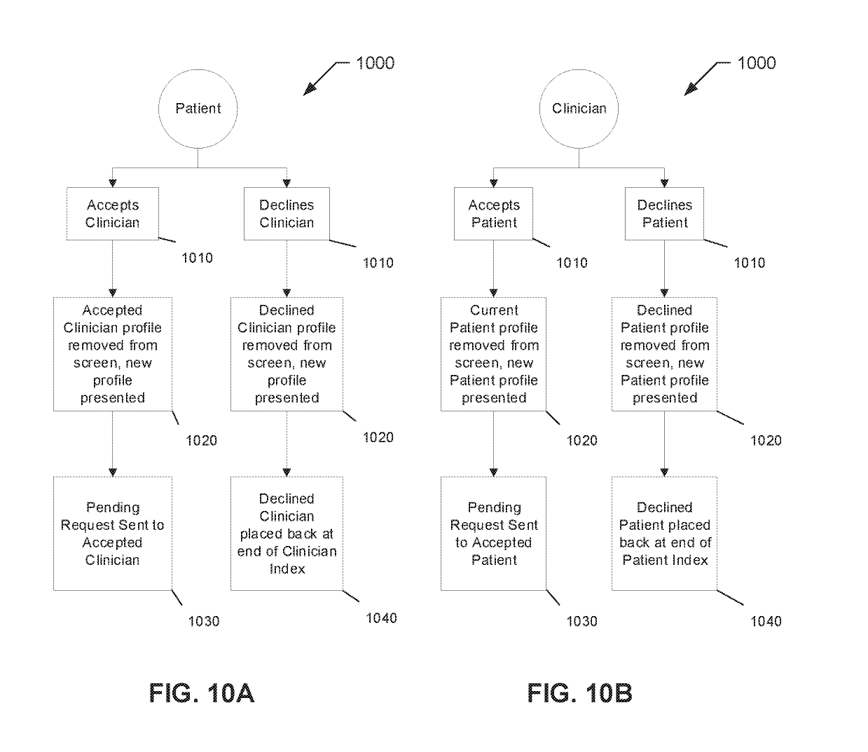

The healthcare provider and patient indexes 610 and 618 enable the match generator 210 to display potential matches to patients and/or healthcare providers. In at least one embodiment, after the match generator 210 determines the healthcare provider index 610, the match generator 210 may generate the healthcare provider profile cards 800 for sending to the application 120 for display. A healthcare provider profile card 800 may be displayed individually on a screen of the patient terminal 110 through the application 120. A patient may then view the healthcare provider profile cards 800 and make an indication to accept or decline a healthcare provider in each healthcare provider profile card 800 as is discussed in more detail with regard to FIG. 10.

Each patient and healthcare provider profile card 900 and 800 includes a subset or portion of a patient and healthcare provider profile record 608 and 612 respectively. For example, a patient profile card 900 may include a name, picture, age, gender, city of residence, medical care requested, availability, and any soft information provided by the patient such as hobbies, movie quote, or something about themselves. A healthcare provider profile card 800 may include a name, picture, city of practice, medical licenses, medical specialties, availability (as determined from a schedule at the registration module 206 or the healthcare provider server 104), and any soft information such as favorite quotes, hobbies, etc. In some embodiments, a healthcare provider profile card 800 may include a section for displaying multimedia content or a static advertisement. For example, the section may include a video player is configured to play a brief video provided by the healthcare provider. In some embodiments, the healthcare provider profile card 800 is configured to automatically play the content upon the card being displayed or enable a patient to select if the content is to be played.

In some embodiments, the patient and healthcare provider profile cards 900 and 800 may be dynamic. For example, the registration module 206 or match generator 210 may create a patient or healthcare provider profile card 900 or 800 that includes some information particular to a potential match between a patient and a healthcare provider, but that would not be displayed if a different patient or healthcare provider was viewing the card. For example, if a patient has a strong preference for a healthcare provider that speaks French, and a healthcare provider speaks French, the healthcare provider profile card 800 the patient views for that healthcare provider may include information indicative that the healthcare provider speaks French. However, for other patients without a language preference, the registration module 206 or the match generator 210 may be configured to not provide the French language indication because it is not important to the patient, as determined from the patient's profile record 608.

FIGS. 8A, 8B, and 8C illustrate an example embodiment of a healthcare provider profile card 800 a patient views to decide whether to accept or decline a healthcare provider. FIG. 8A illustrates an example healthcare provider profile card 800 which includes a healthcare provider name field 802, a healthcare provider picture field 804, a healthcare provider address field 824, a healthcare provider fee range field 822, and a healthcare provider rating field 806. In some instances, some of the information on the healthcare provider profile card 800 is interactive, such as the healthcare provider address field 824, which may link to a map program on the patient terminal 110. The healthcare provider profile card 800 may also include an accept button 808 and a decline button 810 that a patient may press to either accept or decline the healthcare provider, and a view details button 812 that a patient may press to view another screen with additional details about the healthcare provider. FIG. 8B illustrates an example embodiment of such a screen with additional healthcare provider details, such as a healthcare provider insurance preference field 826 and a distance away field 814. Healthcare provider profile card 800, in one embodiment, may include a chat button 816 that activates after a patient and healthcare provider match. In other embodiments, chat button 816 may not appear until a patient and healthcare provider match. In at least one embodiment, healthcare provider card 800 may also include a report button 820 that a patient may press to report a healthcare provider, such as an inappropriate or offensive healthcare provider. FIG. 8C illustrates an example embodiment of a healthcare provider information screen 818 that displays even more information about a healthcare provider that a patient may see. The healthcare provider information shown in FIG. 8C may be displayed responsive to a patient selecting an icon on the card 800 of FIG. 8A or 8B or selecting the card 800 itself.

FIGS. 9A, 9B, 9C, and 9D illustrate an example embodiment of a patient profile card 900 a healthcare provider sees to decide whether to accept or decline a patient. FIG. 9A illustrates an example patient profile card 900 which includes a patient name field 912, a patient picture field 914, a patient fee range field 916, a patient symptoms field 918, a patient rating field 902, and a patient address field 904. In some instances, some of the information on the patient profile card 900 is interactive, such as the patient address field 904, which may link to a map program on the patient terminal 110. The patient profile card 900 may also include an accept button 808 and a decline button 810 that a healthcare provider may press to either accept or decline the patient, and a view details button 812 that a healthcare provider may press to view another screen with additional details about the patient.

FIG. 9B illustrates an example embodiment of such a screen with additional patient details, such as a patient insurance field 920 and a distance away field 922. Patient profile card 900, in one embodiment, may include a chat button 924 that activates after a patient and healthcare provider match. In other embodiments, chat button 924 may not appear until a patient and healthcare provider match. In at least one embodiment, patient profile card 900 may also include a report button 906 that a healthcare provider may press to report a patient, such as an inappropriate or offensive patient. FIG. 9C illustrates an example embodiment of a patient information screen 908 that displays even more information about a patient that a healthcare provider may see, including an appointment history button 910 that a healthcare provider may press to view a patient's history of appointment scheduling with various healthcare providers. FIG. 9D is an example embodiment of a screen a healthcare provider may see when viewing a patient's appointment history.

FIGS. 10A and 10B illustrate an example embodiment of a method 1000 of the present disclosure. The method 1000 may be implemented on a computer system, such as the management server 102, the patient terminal 110, and/or the healthcare provider terminal 112. For example, the method 1300 may be implemented by the registration module 206, the match generator 210, the match manager 212, and/or the referral module 214 of the management server 102. The method 1000 may also be implemented by a set of instructions stored on a computer readable medium that, when executed by a processor, cause the computer system to perform the method. For example, all or part of the method1000 may be implemented by the CPU 224 and the memory 226. Although the examples below are described with reference to the flowchart illustrated in FIGS. 10A and 10B, many other methods of performing the acts associated with FIGS. 10A and 10B may be used. For example, the order of some of the blocks may be changed, certain blocks may be combined with other blocks, one or more of the blocks may be repeated, and some of the blocks described may be optional.

It should be appreciated that the method 1000 applies equally to both patients and healthcare providers, as illustrated in FIGS. 10A and 10B, even though the example embodiment is explained below only from a patient's perspective. When a patient views a healthcare provider profile card 800 on a screen of the patient terminal 110 through the application 120, the patient may either accept the healthcare provider by making a first input via the patient terminal 110 or decline the healthcare provider by making a second input via the terminal 110 (step 1010). In some embodiments, the application 120 may be configured to enable a patient to skip a healthcare provider profile card 800 if a decision cannot be made.

The first input may be a screen swipe in a first direction (e.g., up, down, left, right) while a second input may be a screen swipe in a second direction. The first and second inputs may also include selection of a corresponding graphical icon or button (e.g. accept and decline buttons 808 and 810 in FIG. 8). The first and second inputs may further include a gesture made by moving the patient terminal 110, such as pulling the patient terminal 110 towards the patient for a first input and pushing the patient terminal 110 downward towards the floor for the second input. The first and second inputs may also be an affirmative voice command for the first input and a negative voice command for the second input.

After the patient accepts or declines a healthcare provider profile card 800, the application 120 may remove the accepted or declined healthcare provider profile card 800 from the screen and display the next healthcare provider profile card 800 (step 1020). The application 120 may record a patient's response for each healthcare provider profile card (e.g., accept, decline, skip), and transmit the response to the match generator 210. If the patient accepted a healthcare provider profile card 800, the match generator 210 may determine if the corresponding healthcare provider as already accepted the patient. If one has not yet been received, the match generator 210 may send a pending request to the accepted healthcare provider (step 1030). This may include sending a message, such as a text message or a message through the application 120. This may also include updating a pending request interface of the application 120. In some embodiments, the match generator 210 sends pending requests to the appropriate healthcare providers for their response or provides a list of pending requests for viewing in the application 120 on the healthcare provider terminal 112. Each pending request may be a line entry in a table added by the application 120 to a user interface displayed on the healthcare provider terminal 112.

If the patient declined the healthcare provider profile card 800, the application 120 may send the declined healthcare provider profile record 612 to the end of the healthcare provider index 610 (step 1040). In such embodiments, the patient may then be shown the declined healthcare provider profile card 800 again after the patient makes a decision on all of the healthcare provider profile cards 800 the patient has yet to see. In other embodiments, the application 120 may send the declined healthcare provider profile record 612 back to the healthcare provider profile database 216 and remove it from the healthcare provider index 610.

The match generator 210 may use the information from the application 120 to determine if additional cards are to be provided to the application 120 for sequential display. In some embodiments, the application 120 may continue sequencing through healthcare provider profile cards 800 until a patient stops providing responses or the healthcare provider index 610 has been exhausted. In some embodiments, after detecting a patient has reached the end of the healthcare provider index 610, the match generator 210 may determine a second set of potential healthcare provider matches, albeit with lower matching scores, for display to the patient (e.g., healthcare providers that don't match every category of information for a patient).

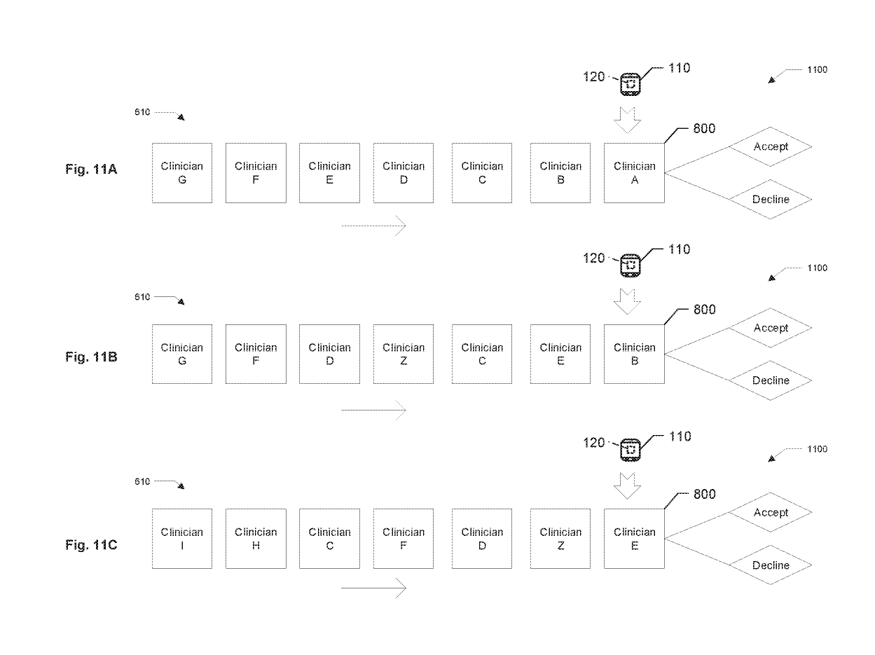

In some embodiments, as illustrated in FIGS. 11A, 11B, and 11C, the match generator 210 may use feedback of acceptances and/or declines to update weights, conditional filters, etc. for refining the matching process in example method 1100. For example, after a patient accepts a healthcare provider or group of healthcare providers, the match generator 210 may determine correlations between patient and healthcare provider info. Weights for these correlations may be increased to reorder the healthcare provider index 610 or to locate other healthcare providers with similar backgrounds or attributes and add those healthcare provider profile records 612 to the current healthcare provider index 610, if they are not already included, or to future healthcare provider indexes 610. In another example, after a patient declines a certain healthcare provider or group of healthcare providers, the match generator 210 may determine correlations between patient and healthcare provider info. Weights for these correlations may be reduced or conditional filters created to reorder the healthcare provider index 610 or to remove healthcare provider profile records 612 with similar information from the current healthcare provider index 610 or from future healthcare provider indexes 610.

FIG. 11A illustrates an example healthcare provider index 610 comprising an order of healthcare provider profile cards 800 for Healthcare providers A, B, C, D, E, F, and G that move in the direction of the arrow to be displayed on patient terminal 110. In some embodiments, more than one healthcare provider profile card 800 may be displayed at the same time. It should be understood that the healthcare provider index 610 may comprise many more healthcare provider profile cards 800 and only seven are shown for illustrative purposes. Application 120 is displays the healthcare provider profile card 800 of Healthcare provider A on patient terminal 110 and the patient may choose to accept or decline it. FIG. 11B illustrates an example embodiment of the match generator 210 updating the healthcare provider index 610 after the patient accepts Healthcare provider A in FIG. 11A. The match generator 210 may use the feedback from the acceptance of Healthcare provider A to reorder the healthcare provider index 610 by moving the healthcare provider profile card 800 of Healthcare provider E up in the order as illustrated. The match generator 210 may also locate a healthcare provider profile card 800 of Healthcare provider Z that was not already in the healthcare provider index 610 and add it to the healthcare provider index 610.

FIG. 11C illustrates an example embodiment of the match generator 210 updating the healthcare provider index 610 after the patient declines Healthcare provider B in FIG. 11B. The match generator 210 may use the feedback from the decline of Healthcare provider B to reorder the healthcare provider index 610 by moving the healthcare provider profile card 800 of Healthcare provider C back in the order as illustrated. The match generator 210 may also remove the healthcare provider profile card 800 of Healthcare provider G from the healthcare provider index 610. It should be understood that the healthcare provider profile cards 800 of Healthcare providers H and I were next in line, but not illustrated, in FIGS. 11A and 11B. It should also be appreciated that the match generator 210 may perform the same function for the patient index 618 and patient profile records 608.

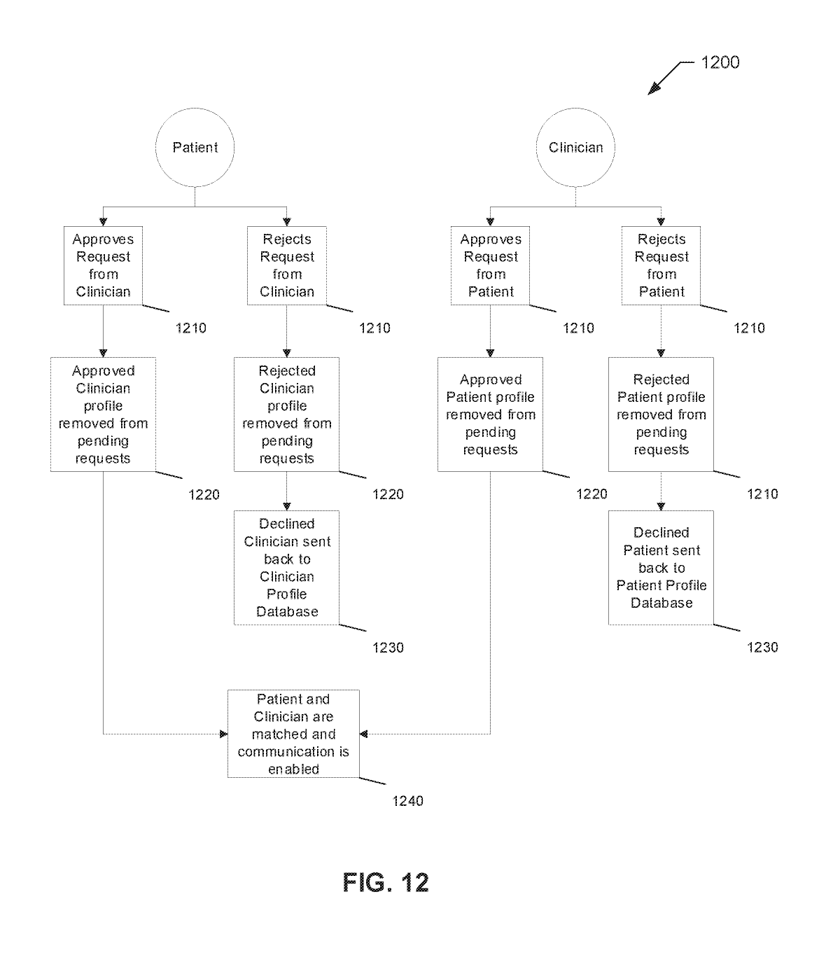

FIG. 12 illustrates an example embodiment of a method 1200 of the present disclosure for processing pending requests. The method 1200 may be implemented on a computer system, such as the management server 102, the patient terminal 110, and/or the healthcare provider terminal 112. For example, the method 1300 may be implemented by the registration module 206, the match generator 210, the match manager 212, and/or the referral module 214 of the management server 102. The method 1200 may also be implemented by a set of instructions stored on a computer readable medium that, when executed by a processor, cause the computer system to perform the method. For example, all or part of the method 1200 may be implemented by the CPU 224 and the memory 226. Although the examples below are described with reference to the flowchart illustrated in FIG. 12, many other methods of performing the acts associated with FIG. 12 may be used. For example, the order of some of the blocks may be changed, certain blocks may be combined with other blocks, one or more of the blocks may be repeated, and some of the blocks described may be optional.

Referring to the example method 1200 illustrated in FIG. 12, either a patient or healthcare provider may have pending requests indicative of an acceptance of the other party. The application 120 may provide a pending requests user interface that provides a list of all pending requests for the patient or healthcare provider to review. The pending requests may include a profile card 900 or 800 of the patient or healthcare provider that provided the initial acceptance. In various embodiments, the patient or healthcare provider progress through a sequence of pending requests providing their own approvals or rejections (step 1210). Regardless of the decision, when a patient or healthcare provider chooses to approve or reject a pending request, the corresponding healthcare provider or patient profile record 612 or 608 may be removed from the list of pending requests (step 1220).

If the match generator 210 receives a rejection indication for a pending request, the match generator 210 may send the rejected patient or healthcare provider profile record 608 or 612 back to its respective patient or healthcare provider profile database 208 or 216 (step 1230). In some embodiments, the match generator 210 may send the rejected patient or healthcare provider profile record 608 or 612 to the back of the patient or healthcare provider index 618 or 610, respectively. In some embodiments, the match generator 210 may send the rejected party, via the application 120, a message indicative that their pending request was rejected. In other embodiments, the match generator 210 only removes the pending request from a list of pending requests for the rejected party.

If the match generator 210 receives an approval indication for a pending request, the match generator 210 may be configured to designate the accepting party and the requesting party as a match and transmit the information to the example match manager 212 (step 1240). This process may include providing an acceptance index for the patient and healthcare provider with an identifier of the other indicative of the match. Upon receiving an indication of a match, the match manager 212 may transmit the information to the example communication manager 220 to enable the matched patient and healthcare provider to communicate, as discussed in more detail below. In various embodiments, the match manager 212 also transmits the information to the example schedule module 222 to enable the matched patient to schedule an appointment on the matched healthcare provider's calendar, which will also be discussed in more detail below.

The match manager 212 may be configured to manage matches between patients and healthcare providers after they occur. For example, the application 120 and/or the match manager 212 may maintain a list or index of matches of healthcare providers for each patient, or vice versa. In addition, after a patient and healthcare provider are matched, if a patient no longer desires to be matched with the healthcare provider, the patient may make such an indication on the application 120. Accordingly, the match manager 212 may update the match list and/or index to reflect that the patient declined the match. The match manager 212 may also transmit the information of the match removal to the communication manager 220 and/or schedule module 222 to prevent communication between the parties going forward and to prevent the patient from scheduling an appointment with the healthcare provider.

### Referral System Embodiment

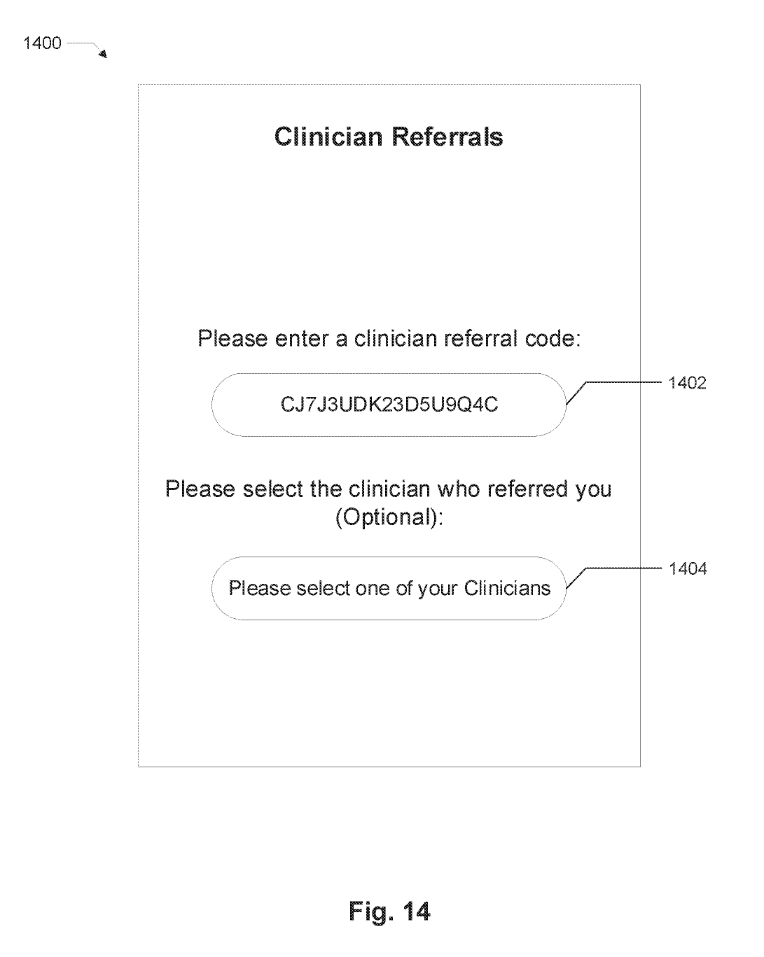

In various embodiments of the system 100, patients may also find and/or match with healthcare providers through the use of a referral code 1402 (FIG. 14). Referral codes 1402 may be identifiers, such as a sequence of letters, numbers, and/or symbols, that are uniquely associated with and identify a particular healthcare provider. In some embodiments, a patient can input a referral code 1402 to directly locate a particular healthcare provider and decide whether to accept or decline the healthcare provider. This avoids the patient having to wait to come across the particular healthcare provider when sequencing through the healthcare provider profile cards 800 in the healthcare provider index 610. In other embodiments, inputting a referral code 1402 may directly match a patient with a healthcare provider without the healthcare provider having to approve a request from the patient. In some embodiments, when a patient inputs a referral code 1402 for a referred healthcare provider, the healthcare provider profile card 800 of the referred healthcare provider includes a designator indicating that the healthcare provider was referred.

The example referral module 214 of the system 100 may be configured to store and analyze the referral codes 1402 for all healthcare providers. For example, the referral codes 1402 and their associated healthcare providers may be stored in a look-up table. In various embodiments, a patient may enter a referral code 1402 via the application 120, which transmits the referral code 1402 to the referral module 214. The referral module 214 may identify the associated healthcare provider using the referral code 1402 in a look-up table, locate the healthcare provider's profile record 800 in the healthcare provider profile database 216 and transmit the healthcare provider profile record 800 to the application 120 for the application 120 to display the healthcare provider's profile card 800 to the patient. In other embodiments, the referral codes 1402 may be stored directly on the application 120 and entering a referral code 1402 on the application 120 directly causes the application 120 to display the corresponding healthcare provider. Once the patient is viewing the healthcare provider's profile card 800 associated with the referral code 1402, the patient may choose to accept or decline the healthcare provider as described in more detail above.

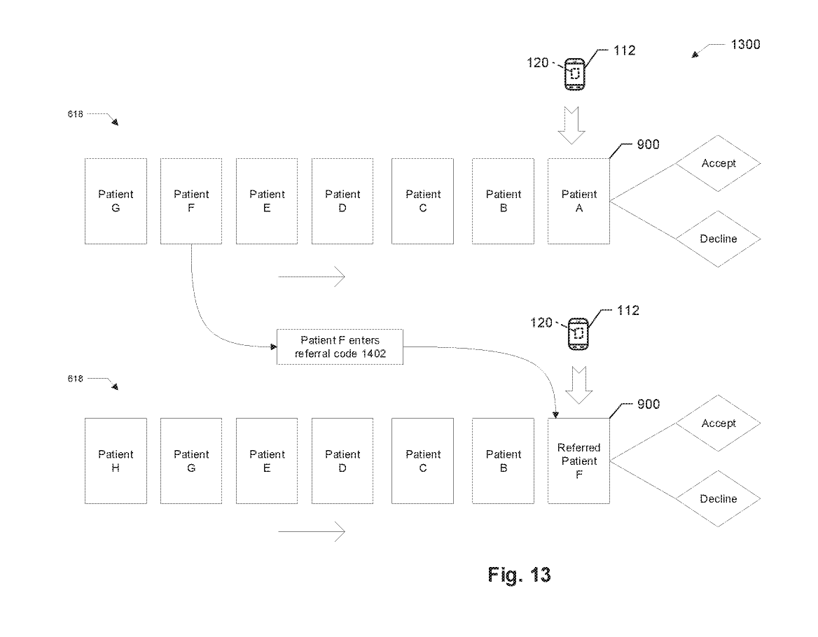

In some embodiments, as illustrated in example method 1300 in FIG. 13, if a patient accepts a healthcare provider using a referral code 1402, the application 120 or match generator 210 will move the patient's profile card 900 to the top of the healthcare provider's patient index 618. For example, the method 1300 illustrates an example patient index 618 comprising an order of patient profile cards 900 for Patients A, B, C, D, E, F, and G that move in the direction of the arrow to be displayed on healthcare provider terminal 112. It should be understood that the patient index 618 may comprise many more patient profile cards 900 and only seven are shown for illustrative purposes. Application 120 is displaying the patient profile card 900 of Patient A on healthcare provider terminal 112 and the healthcare provider may choose to accept or decline it. Once the healthcare provider makes a choice, the next patient profile card 900 in line for the application 120 to display is that of patient B. In this example, however, Patient F enters a referral code 1402 for the healthcare provider and accepts the healthcare provider, and thus is moved to the top of the patient index 618 to be displayed on the healthcare provider terminal 112.

It should be understood that the method 1300 may be implemented on a computer system, such as the management server 102, the patient terminal 110, and/or the healthcare provider terminal 112. For example, the method 1300 may be implemented by the registration module 206, the match generator 210, the match manager 212, and/or the referral module 214 of the management server 102. The method 1400 may also be implemented by a set of instructions stored on a computer readable medium that, when executed by a processor, cause the computer system to perform the method. For example, all or part of the method 1300 may be implemented by the CPU 224 and the memory 226. Although the examples below are described with reference to the flowchart illustrated in FIG. 13, many other methods of performing the acts associated with FIG. 13 may be used. For example, the order of some of the blocks may be changed, certain blocks may be combined with other blocks, one or more of the blocks may be repeated, and some of the blocks described may be optional.

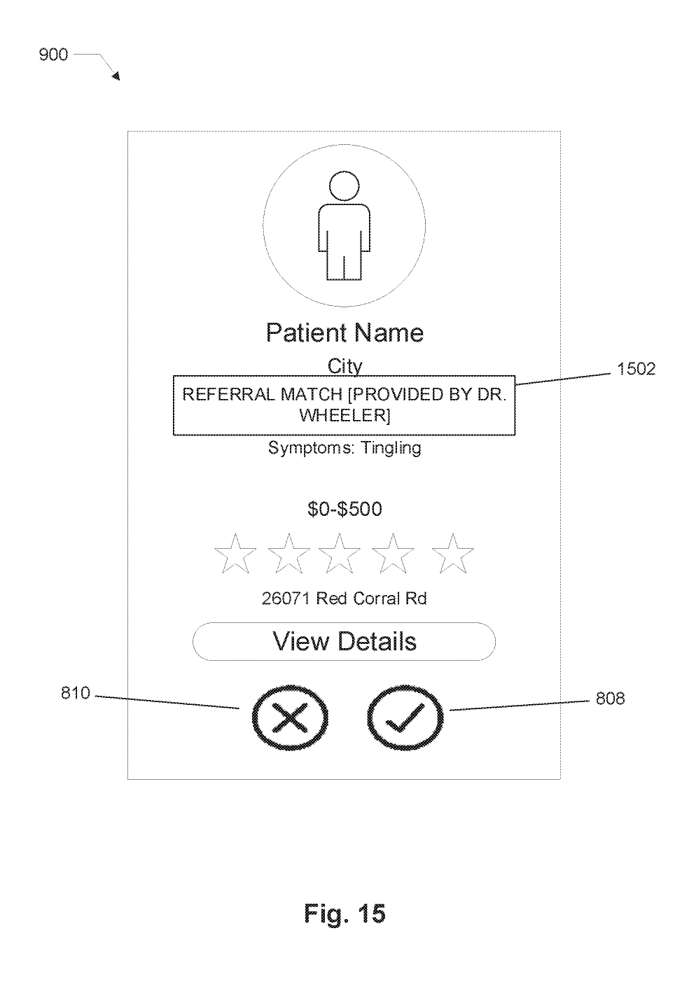

FIG. 14 illustrates an example embodiment of a patient referral screen 1400 displayed on the patient terminal 110 at which a patient may enter a healthcare provider referral code 1402. The example patient referral screen 1400 includes an input for a referral code 1402 and an input for a referring healthcare provider 1404. In various embodiments as illustrated in FIG. 15, when a patient inputs a referral code 1402, the patient's profile card 900 will include a referral match indication 1502. The referral match indication 1502 may indicate the referring healthcare provider if the patient chooses to provide it. The referral match indication 1502 indicates to the healthcare provider viewing the patient profile card 900 that this patient was specifically referred to the healthcare provider and may make the healthcare provider more likely to accept the patient. In some embodiments, the match generator 210 and/or the match manager 212 may move a patient profile card 900 to a front of an order of a patient index 618 for a referred healthcare provider regardless of other matching criteria.

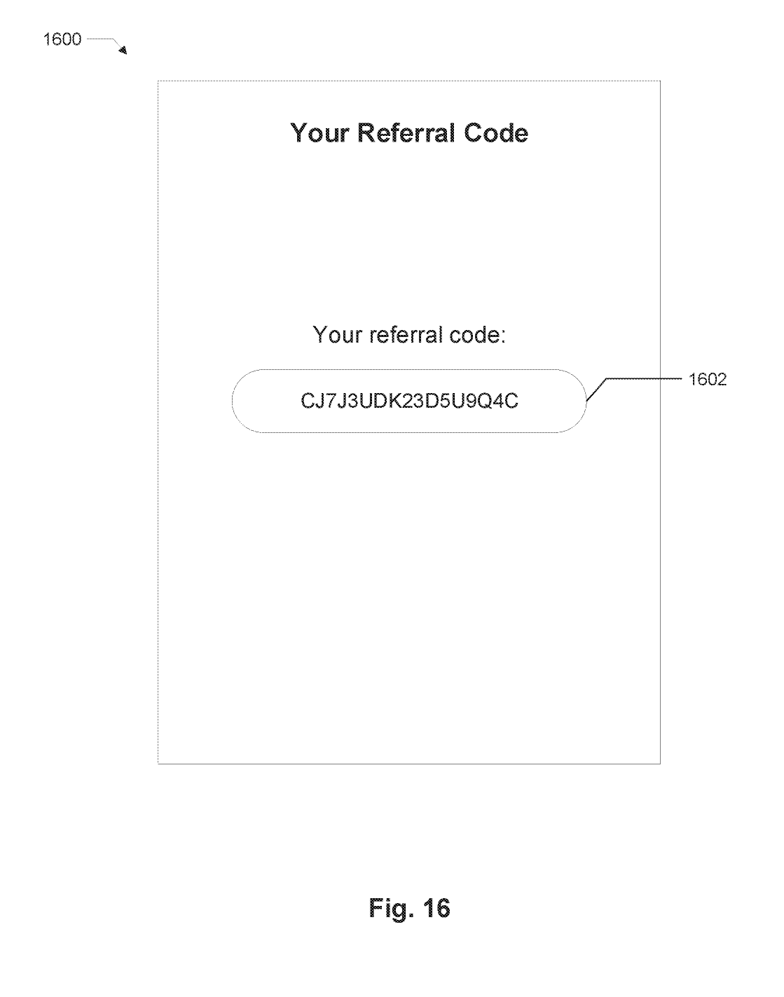

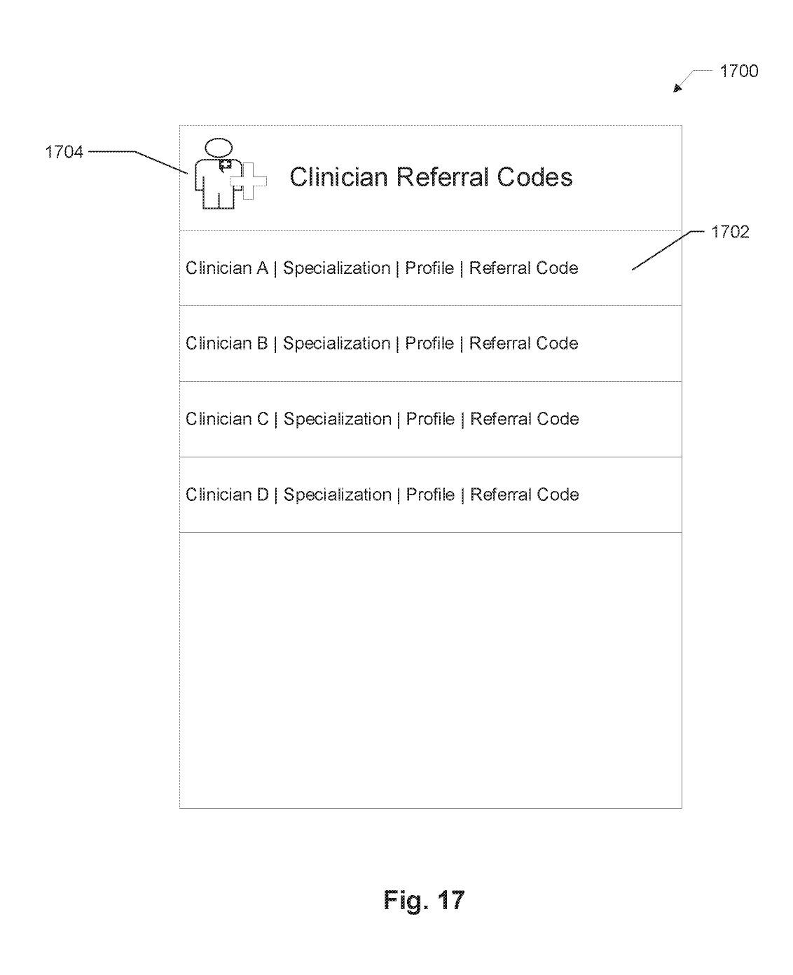

In embodiments that utilize referral codes 1402, a healthcare provider may be able to locate the healthcare provider's own referral code via the healthcare provider terminal 112 and the application 120. FIG. 16 illustrates an example embodiment of a healthcare provider referral code screen 1600 the application 120 may display on healthcare provider terminal 112 for healthcare providers to view their own referral code 1402. Healthcare providers being aware of their own referral code 1402 enables healthcare providers to share it with patients so patients can share the referral code 1402 with other potential patients, who can then input it on the application 120 to locate the healthcare provider. For example, healthcare providers may be able to send their code to the messaging section of matched patients on the application 120. Healthcare providers may also share their referral code 1402 with other healthcare providers so those other healthcare providers can share it with their patients. FIG. 17 illustrates an example embodiment of a saved referral codes screen 1700 the application 120 may display at which a healthcare provider can add and store other healthcare providers' referral codes for easy future reference. The example saved referral codes screen 1700 includes a plurality of referral codes 1402 and an add referral button 1704. In some embodiments, healthcare providers may be able to view the healthcare provider profile cards 800 of the healthcare providers whose referral codes 1402 they have saved.

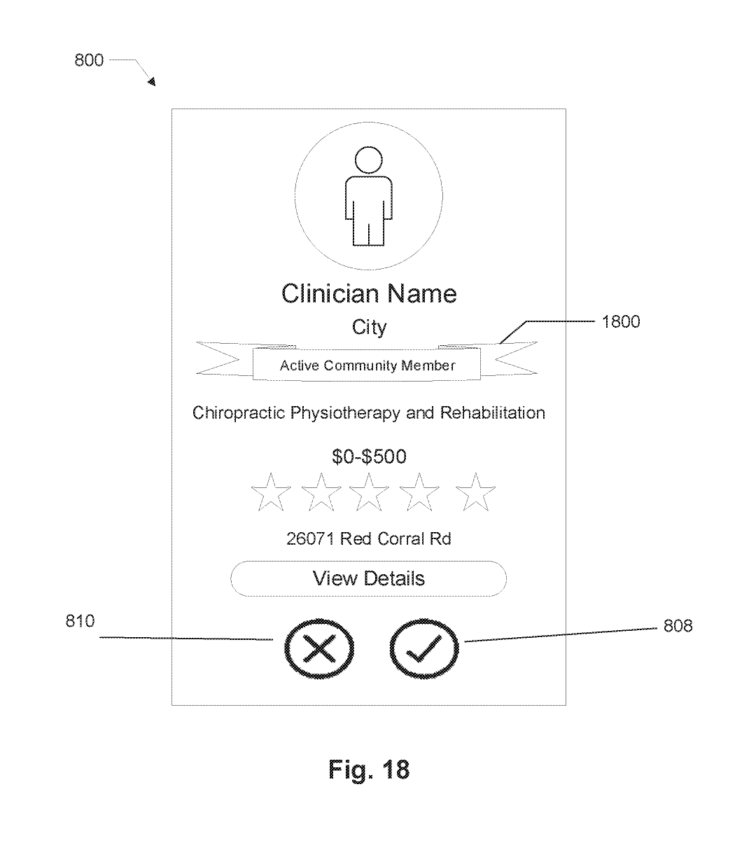

In some embodiments, healthcare providers may receive incentives based on their performance behavior. For example, healthcare providers who have been active in the community by continually referring patients to other healthcare providers and have received good feedback and ratings from their own patients, along with other performance metrics, may receive incentives in the application 120. Incentives may include boosted profile view priority, free subscriptions, badges of recognition, and other potential incentives to make healthcare providers stand out to patients in the application 120. FIG. 18 illustrates an example embodiment of a healthcare provider profile card 800 that includes a badge 1800 that indicates the healthcare provider has been active in referring other healthcare providers.

### Communication between Healthcare provider and Patient Embodiment

In various embodiments, once a patient and healthcare provider are matched, they are able to communicate with one another. The example communication manager 220 may be configured to enable patients and healthcare providers to communicate with each other via the application 120 on their terminals 110 and 112. For example, patients may select a match from their match list, which causes the application 120 to display the corresponding healthcare provider profile card 800. In some embodiments, the application 120 displays buttons or options for contacting the healthcare provider on the healthcare provider profile card 800. For example, a chat button 816 (FIGS. 8 and 9) may be activated, or may appear, on the healthcare provider profile card 800 once a patient and healthcare provider are matched. In another example, selection of a telephone button may cause the application 120 to activate a telecommunications application on the terminal 110 to place a call to the healthcare provider. In another example still, selection of a text button may cause the application 120 to activate a SMS or text-based program for sending a message to the healthcare provider. The message may be contained or provided by the application 120 or another third-party based application.

The communication manager 220 may provide or facilitate text-based messaging, picture sharing, telecommunication, live-video sharing, emoji-based symptoms or conditions, etc. In some embodiments, the registration module 206 enables patients and healthcare providers to specify any contact preferences. For example, a healthcare provider may only accept email and text messages. Accordingly, the communication manager 220 only enables patients via the application 120 to contact that healthcare provider through email or text.

In some embodiments, the communications between the patient and healthcare provider may be encrypted by the application 120. For example, the communications may be encrypted in accordance with HIPAA standards. In some instances, communications may be routed through the communication manager 220, which operates as a router or switch. For example, communications from the application 120 may be addressed to the communication manager 220, which uses a routing or look up table to change the destination to the intended terminal 110 or 112. The messages may include a header field that identifies the intended recipient (e.g., “USER123”), which the communication manager 220 uses for determining a destination address.

In other embodiments, the communication manager 220 is configured to send messages to the application 120 that enable the terminals 110 and 112 to communicate directly with each other (over a network). After receiving a message from the communication manager 220, the application 120 updates an address list to provide a destination address for the matched patient or healthcare provider. The application 120 uses the provided address to send communications to the application 120, or more generally, the matched terminal 110 or 112.

### Appointment Scheduling Embodiment

In various embodiments, once a patient and healthcare provider are matched, a patient is able to directly schedule an appointment with the healthcare provider via the application 120. The example schedule module 222 is configured to enable a matched patient to book an appointment with a healthcare provider. A healthcare provider is able to set the healthcare provider's availability for appointments via the application 120, which may then be transmitted to the schedule module 222. The schedule module 222 may maintain a local schedule for each healthcare provider. Alternatively, the schedule module 222 may access a schedule located at the healthcare server 104 or 106 associated with the healthcare provider and/or a schedule application (or through the application 120) on the healthcare provider terminal 112.

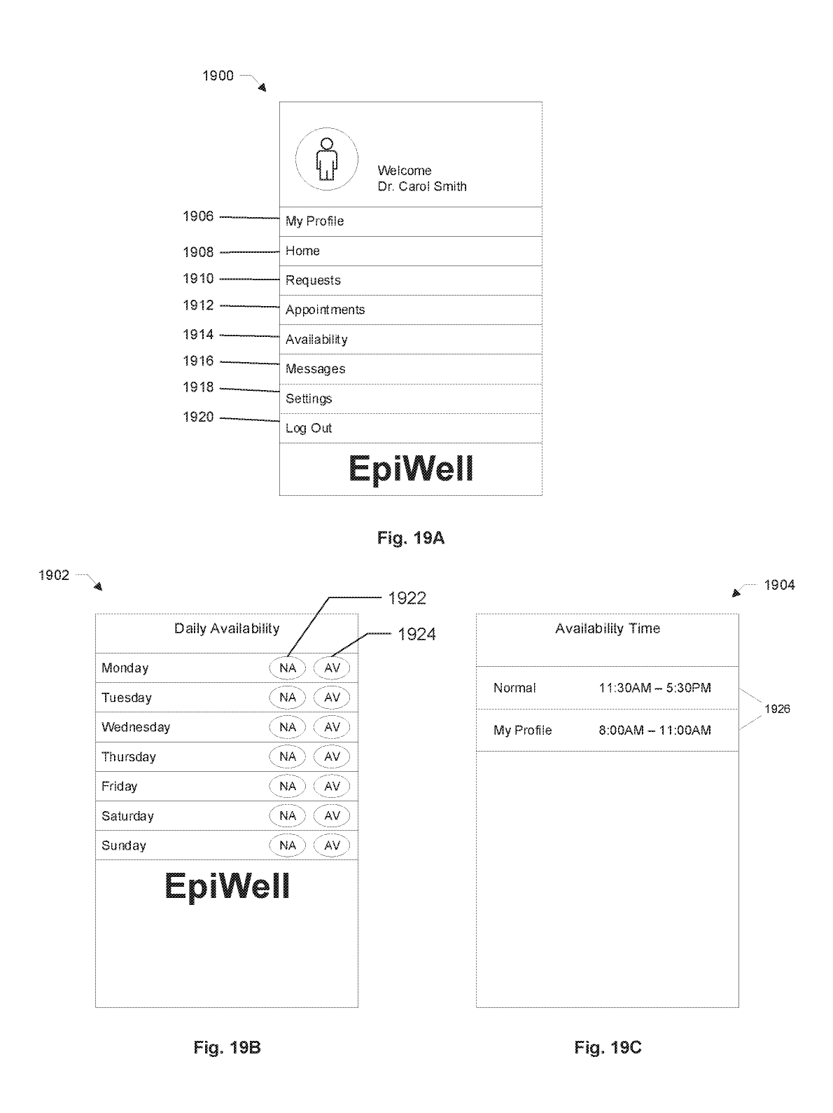

FIGS. 19A, 19B, and 19C illustrate example embodiments of display screens of the application 120 for a healthcare provider to set availability. FIG. 19A illustrates an example healthcare provider menu screen 1900 which includes a profile button 1906, a home button 1908, a requests button 1910, an appointments button 1912, an availability button 1914, a messages button 1916, a setting button 1918, and a log out button 1920. In one embodiment, if the healthcare provider selects the availability button 1914, the application 120 may direct the healthcare provider to an example availability screen 1902 as illustrated in FIG. 19B. For each day of the week, the healthcare provider may select a not available (NA) button 1922 or an available (AV) button 1924. If the healthcare provider selects the available (AV) button 1924 for a day, then in one embodiment, the application 120 may direct the healthcare provider to an example availability time screen 1904 as illustrated in FIG. 19C. A healthcare provider may edit the available time 1926 to set when patients can schedule appointments on any given day. It should be appreciated that in other embodiments a healthcare provider may set availability in other manners and the application 120 may display different screens.

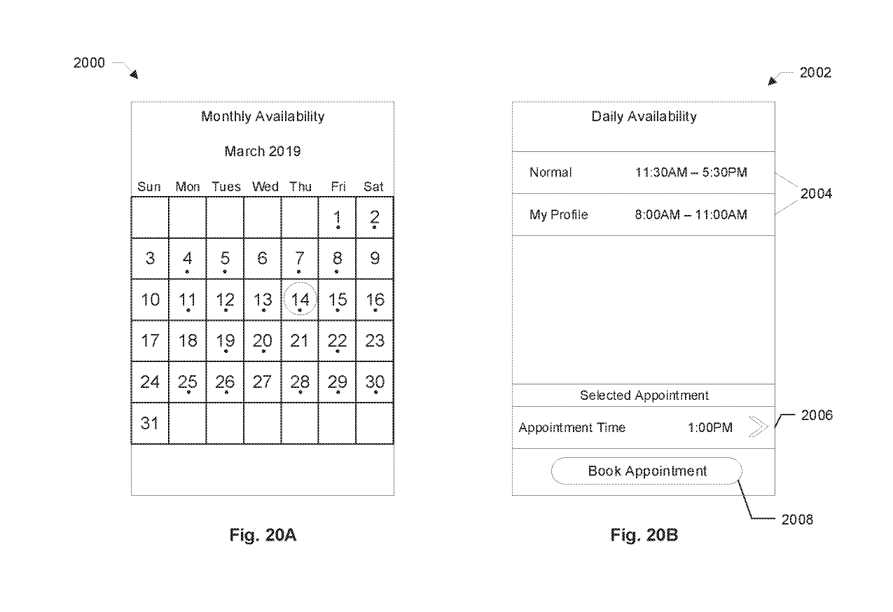

Once a healthcare provider has set availability for appointments, a patient can view a healthcare provider's availability and directly set an appointment with the healthcare provider. FIGS. 20A and 20B illustrate example embodiments of display screens of the application 120 for patients to schedule appointments. FIG. 20A illustrates an example monthly availability screen 2000 which displays a calendar month of days. In various embodiments, monthly availability screen 2000 includes indications for each day whether there are available time slots on that day. For example, days with availability for appointments may have a green dot and days without availability may have a red dot or no dot at all. In one embodiment, when a patient uses the application 120 to select a day with an available time slot (e.g., March 14th is selected in FIG. 20B) application 120 may direct the patient to example scheduling screen 2002 as illustrated in FIG. 20B. Scheduling screen 2002 displays available appointment times 2004. The patient is then able to select an appointment time 2006 and press the book appointment button 2008 to schedule an appointment, which also causes the schedule module 222 to update the healthcare provider's schedule.

In some embodiments, the schedule module 222 sends a message to the healthcare provider terminal 112 and/or the server 104 and 106 indicative of the selected time slot to enable a local calendar to be updated automatically. In addition, the schedule module 222 may transmit a portion of the information the patient provided at registration, which may be used by the server 104 or 106 to create a patient medical record or patient registration form.

### Medical Diagnostic Tests Embodiment

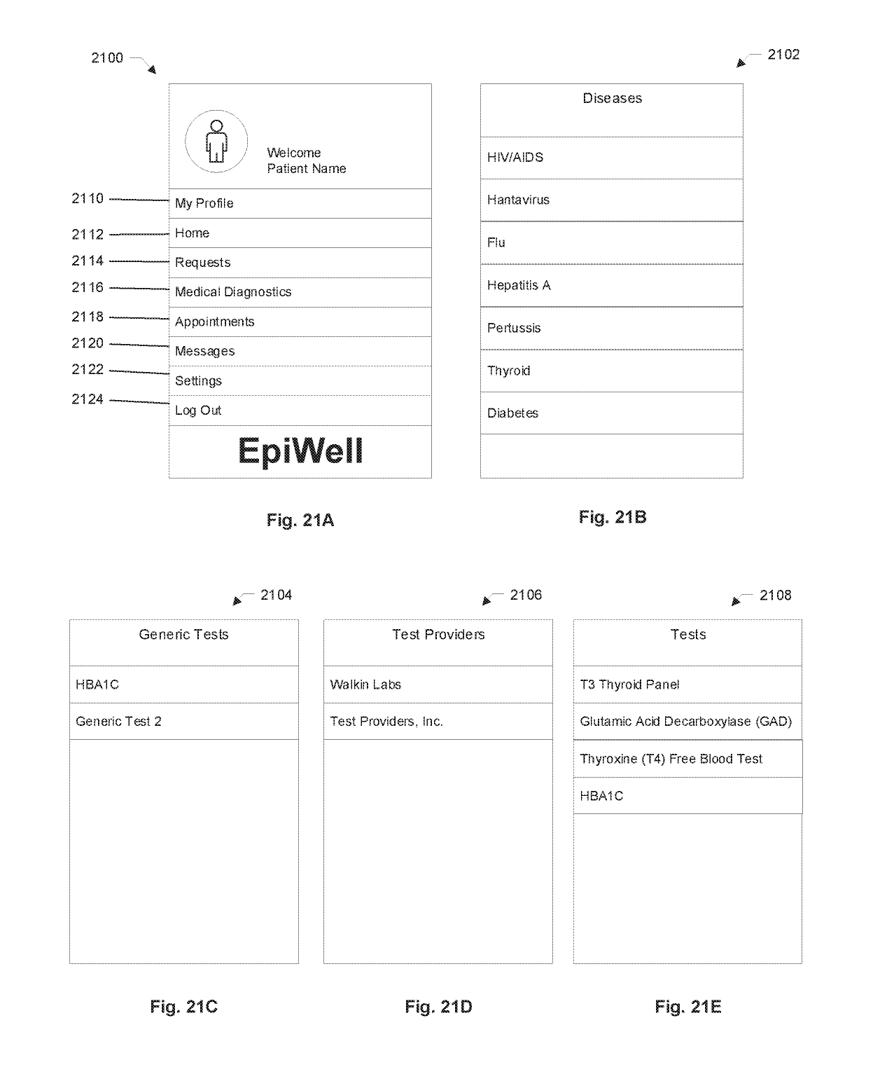

In some embodiments, the schedule module 222 is also configured to enable patients to schedule their own medical diagnostic tests. In some embodiments, patients may schedule medical diagnostic tests through affiliate provider links on the application 120 which may take patients to an affiliate provider's website for patients to schedule appointments with the affiliate provider. In some embodiments, patients may alternatively or additionally schedule medical diagnostic tests directly on the application 120. FIGS. 21A, 21B, 21C, 21D, and 21E illustrate example embodiments of screens the application 120 may display to a patient as the patient schedules a medical diagnostic test. FIG. 21A illustrates a patient menu screen 2100 which includes a profile button 2110, a home button 2112, a requests button 2114, a medical diagnostics button 2116, an appointments button 2118, a messages button 2120, a settings button 2122, and a log out button 2124. In at least one embodiment, if a patient selects the medical diagnostics button 216, the application 120 will direct the patient to the diseases screen 2102 as illustrated in FIG. 21B. At the diseases screen 2102, the patient may select for which disease they would like to schedule a medical diagnostic test. For example, diseases may include HIV/AIDS, Hantavirus, Flu, Hepatitis A, Pertussis, Thyroid, Diabetes, among other diseases. In at least one embodiment, after the patient selects a disease, the application 120 may direct the patient to the generic tests screen 2104 as illustrated in FIG. 21C. At the generic tests screen 2104, patients may select a category of tests pertaining to the disease they selected. For example, if the patient selected Diabetes, the patient could then select HBA1C as a generic category of tests.

In at least one embodiment, after the patient selects a category of tests, the application 120 may direct the patient to the test providers screen 2106 as illustrated in FIG. 21D. At the test providers screen 2106, patients may select the provider they want to go to for performing the medical diagnostic test. For example, a provider may be a hospital, a healthcare provider's office, an independent medical diagnostics testing company, or any other medically licensed entity that provides medical diagnostic tests. In at least one embodiment, after the patient selects a test provider, the application 120 may direct the patient to a tests screen 2108 as illustrated in FIG. 21E. At the test screen 2108, patients may select a specific medical diagnostic test that they want to schedule. For example, tests could include a t3 Thyroid Panel test, a Glutamic Acid Decarboxylase Antibody test, a Thyroxine (T4) Free Blood Test, a HBA1C test, among many other types of medical diagnostic tests. In at least one embodiment, after the patient selects a test, the application 120 prompts the patient to select a date and time, confirms the scheduling with the patient, and then updates the patient's appointment schedule. In other embodiments, after the patient selects a test, the application 120 may direct the patient to a third-party application or website, such as the test providers website, to complete the scheduling.

### Conclusion

Without further elaboration, it is believed that one skilled in the art can use the preceding description to utilize the claimed inventions to their fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles discussed. In other words, various modifications and improvements of the embodiments specifically disclosed in the description above are within the scope of the appended claims. For example, any suitable combination of features of the various embodiments described is contemplated. The scope of the invention is therefore defined by the following claims.

All of the disclosed methods and procedures described in this disclosure can be implemented using one or more computer programs or components. These components may be provided as a series of computer instructions on any conventional computer readable medium or machine readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

1. A patient-healthcare provider matching apparatus comprising:
a processor;
a patient database including patient profile records for a plurality of patients, each patient profile record including patient information provided by a respective patient;
a healthcare provider database including healthcare provider profile records for a plurality of healthcare providers, each healthcare provider profile record including healthcare provider information provided by a respective healthcare provider;
a linkage database including records indicative of patient acceptances of healthcare providers, healthcare provider acceptances of patients, and linkages between mutually accepted patients and healthcare providers; and
a memory storing instructions, which when executed by the processor, cause the processor to provide for matching between a patient and a healthcare provider by:
for the patient, comparing the patient profile record to the healthcare provider profile records in the healthcare provider database to determine a healthcare provider index of potential matching healthcare providers,
creating or accessing a healthcare provider profile card for each of the potential matching healthcare providers,
determining an order for the healthcare provider profile cards based on a degree of matching with the patient profile record, with healthcare provider profile cards having a higher degree of matching being placed first in the order,
causing a first ordered healthcare provider profile card to be displayed at a patient terminal of the patient,
receiving, from the patient terminal, a patient input in relation to the first ordered healthcare provider profile card,
responsive to the patient input being indicative of a rejection of the healthcare provider profile card, causing a next ordered healthcare provider profile card to be displayed at the patient terminal,
responsive to the patient input being indicative of an acceptance of the healthcare provider profile card, determining if the healthcare provider has already accepted the patient by accessing the linkage database,
responsive to the healthcare provider having not accepted the patient, transmitting a message to a healthcare provider terminal of the healthcare provider indicative of a pending request from the patient, and
responsive to the healthcare provider having already accepted the patient, causing a linkage record to be created indicative of a pairing between the patient and the healthcare provider and enabling the patient and the healthcare provider to communicate with each other.

2. The apparatus of claim 1, wherein the memory stores additional instructions, which when executed by the processor, cause the processor to provide for matching between the healthcare provider and the patient by:
for the healthcare provider, comparing the healthcare provider profile record to the patient profile records in the patient database to determine a patient index of potential matching patients,
creating or accessing a patient profile card for each of the potential matching patients,
determining an order for the patient profile cards based on a degree of matching with the healthcare provider profile record, with patient profile cards having a higher degree of matching being placed first in the order,
causing a first ordered patient profile card to be displayed at the healthcare provider terminal of the healthcare provider,
receiving, from the healthcare provider terminal, a healthcare provider input in relation to the first ordered patient profile card,
responsive to the healthcare provider input being indicative of a rejection of the patient profile card, causing a next ordered patient profile card to be displayed at the healthcare provider terminal,
responsive to the healthcare provider input being indicative of an acceptance of the patient profile card, determining if the patient has already accepted the healthcare provider by accessing the linkage database,
responsive to the patient having not accepted the patient, transmitting a message to the patient terminal of the patient indicative of a pending request from the healthcare provider, and
responsive to the patient having already accepted the healthcare provider, causing a linkage record to be created indicative of a pairing between the patient and the healthcare provider and enabling the patient and the healthcare provider to communicate with each other.

3. The apparatus of claim 1, wherein the memory stores additional instructions, which when executed by the processor, cause the processor to:
before or during the comparison of the patient profile record to the healthcare provider profile records in the healthcare provider database, access the linkage database to determine healthcare providers that have already rejected the patient; and
remove the determined healthcare providers that have already rejected the patient from being potential matching healthcare providers.

4. The apparatus of claim 1, wherein the memory stores additional instructions, which when executed by the processor, cause the processor to:
receive from the patient device a referral code corresponding to a referred healthcare provider having a healthcare provider profile record in the healthcare provider database;
before or during the comparison of the patient profile record to the healthcare provider profile records in the healthcare provider database, add the healthcare provider profile record of the referred healthcare provider to the healthcare provider index regardless of a comparison of the patient profile record to the healthcare provider profile record of the referred healthcare provider; and
place a healthcare provider profile card of the referred healthcare provider at a front of the order.

5. The apparatus of claim 4, wherein the memory stores additional instructions, which when executed by the processor, cause the processor to cause a referred designator to be displayed on the healthcare provider profile card of the referred healthcare provider.

6. The apparatus of claim 1, wherein the memory stores additional instructions, which when executed by the processor, cause the processor to:
determine at least some patient information of the patient that does not match comparable healthcare provider information of a potential matching healthcare provider; and
remove the non-matching healthcare provider information from the healthcare provider profile card of the potential matching healthcare provider before making the healthcare provider profile card available for display to the patient.

7. The apparatus of claim 6, wherein at least some of the patient information that does not match the comparable healthcare provider information corresponds to a category or a field that is coded as being non-critical.

8. The apparatus of claim 7, wherein the category or field is related to at least one of a language spoken, an ethnicity, a hobby, a favorite television/movie, or a social view.

9. The apparatus of claim 1, wherein the memory stores additional instructions, which when executed by the processor, cause the processor to create the healthcare provider profile cards by selecting a subset of the healthcare provider information for the respective healthcare provider.

10. The apparatus of claim 9, wherein the healthcare provider information includes a healthcare provider name, a healthcare provider picture, a healthcare provider age, a healthcare provider gender, a healthcare provider email, a healthcare provider phone number, a healthcare provider address, a healthcare provider specialization, a healthcare provider fee range, and healthcare provider accepted insurance companies, and a healthcare provider desired distance, and
wherein the healthcare provider profile card includes at least one of a healthcare provider name, a healthcare provider picture, a healthcare provider address, a healthcare provider specialization, a healthcare provider fee range, and healthcare provider accepted insurance companies.

11. The apparatus of claim 1, wherein the memory stores additional instructions, which when executed by the processor, cause the processor to:
determine a rating for a respective healthcare provider for each of the healthcare provider profile cards; and
display the determined rating on the respective healthcare provider profile cards.

12. The apparatus of claim 1, wherein the memory stores additional instructions, which when executed by the processor, cause the processor to at least one of:
after enabling the patient and the healthcare provider to communicate with each other, provide a communication option on profile cards of the matching patient and healthcare provider that activates at least one of a chat session, a live-video session, a telecommunication session, or an email program; or
after enabling the patient and the healthcare provider to communicate with each other, provide contact information on the healthcare provider profile card of the matching healthcare provider or transmit the contact information of the healthcare provider to the patient terminal.

13. The apparatus of claim 1, wherein the memory stores additional instructions, which when executed by the processor, cause the processor to:
after enabling the patient and the healthcare provider to communicate with each other, cause a schedule of the matching healthcare provider to be displayed on the patient terminal;
receive a patient scheduling message indicative of a date and time for reserving an appointment;
add an indication of the appointment to the schedule; and
transmit a healthcare provider scheduling message to be transmitted to the healthcare provider terminal that is indicative of the appointment.

14. The apparatus of claim 1, wherein the memory stores additional instructions, which when executed by the processor, cause the processor to:
responsive to the patient input being indicative of a rejection of the healthcare provider profile card, place the healthcare provider profile card of the rejected healthcare provider into a lower position into the order.

15. The apparatus of claim 1, wherein the patient input includes at least one of a swiping motion, a selection of a graphical icon, or a gesture made by moving the patient terminal.

16. A patient-healthcare provider matching method comprising:
providing a patient database including patient profile records for a plurality of patients, each patient profile record including patient information provided by a respective patient;
providing a healthcare provider database including healthcare provider profile records for a plurality of healthcare providers, each healthcare provider profile record including healthcare provider information provided by a respective healthcare provider;
providing a linkage database including records indicative of patient acceptances of healthcare providers, healthcare provider acceptances of patients, and linkages between mutually accepted patients and healthcare providers; and
providing for matching between a patient and a healthcare provider by:
for the patient, comparing, via a processor, the patient profile record to the healthcare provider profile records in the healthcare provider database to determine a healthcare provider index of potential matching healthcare providers,
creating or accessing, via the processor, a healthcare provider profile card for each of the potential matching healthcare providers,
determining, via the processor, an order for the healthcare provider profile cards based on a degree of matching with the patient profile record, with healthcare provider profile cards having a higher degree of matching being placed first in the order,
causing, via the processor, a first ordered healthcare provider profile card to be displayed at a patient terminal of the patient,
receiving, from the patient terminal, a patient input in relation to the first ordered healthcare provider profile card,
responsive to the patient input being indicative of a rejection of the healthcare provider profile card, causing, via the processor, a next ordered healthcare provider profile card to be displayed at the patient terminal,
responsive to the patient input being indicative of an acceptance of the healthcare provider profile card, determining, via the processor, if the healthcare provider has already accepted the patient by accessing the linkage database,
responsive to the healthcare provider having not accepted the patient, transmitting, via the processor, a message to a healthcare provider terminal of the healthcare provider indicative of a pending request from the patient, and
responsive to the healthcare provider having already accepted the patient, causing, via the processor, a linkage record to be created indicative of a pairing between the patient and the healthcare provider and enabling the patient and the healthcare provider to communicate with each other.

17. The method of claim 16, further comprising providing for matching between the healthcare provider and the patient by:
for the healthcare provider, comparing, via the processor, the healthcare provider profile record to the patient profile records in the patient database to determine a patient index of potential matching patients;
creating or accessing, via the processor, a patient profile card for each of the potential matching patients;
determining, via the processor, an order for the patient profile cards based on a degree of matching with the healthcare provider profile record, with patient profile cards having a higher degree of matching being placed first in the order;
causing, via the processor, a first ordered patient profile card to be displayed at the healthcare provider terminal of the healthcare provider;
receiving, from the healthcare provider terminal, a healthcare provider input in relation to the first ordered patient profile card;
responsive to the healthcare provider input being indicative of a rejection of the patient profile card, causing, via the processor, a next ordered patient profile card to be displayed at the healthcare provider terminal;
responsive to the healthcare provider input being indicative of an acceptance of the patient profile card, determining, via the processor, if the patient has already accepted the healthcare provider by accessing the linkage database;
responsive to the patient having not accepted the patient, transmitting, via the processor, a message to the patient terminal of the patient indicative of a pending request from the healthcare provider; and
responsive to the patient having already accepted the healthcare provider, causing, via the processor, a linkage record to be created indicative of a pairing between the patient and the healthcare provider and enabling the patient and the healthcare provider to communicate with each other.

18. The method of claim 16, further comprising:
receiving, in the processor from the patient device, a referral code corresponding to a referred healthcare provider having a healthcare provider profile record in the healthcare provider database;
before or during the comparison of the patient profile record to the healthcare provider profile records in the healthcare provider database, adding, via the processor, the healthcare provider profile record of the referred healthcare provider to the healthcare provider index regardless of a comparison of the patient profile record to the healthcare provider profile record of the referred healthcare provider; and
placing, via the processor, a healthcare provider profile card of the referred healthcare provider at a front of the order.

19. The method of claim 18, further comprising causing, via the processor, a referred designator to be displayed on the healthcare provider profile card of the referred healthcare provider.

20. The method of claim 16, further comprising:
after enabling the patient and the healthcare provider to communicate with each other, causing, via the processor, a schedule of the matching healthcare provider to be displayed on the patient terminal;
receiving, in the processor, a patient scheduling message indicative of a date and time for reserving an appointment;
adding, via the processor, an indication of the appointment to the schedule; and
transmitting, via the processor a healthcare provider scheduling message to be transmitted to the healthcare provider terminal that is indicative of the appointment.
